(19) United States
(12) Patent Application Publication
(10) Pub. No.: US 2010/0092560 A1
(43) Pub. Date: Apr. 15, 2010

(54) REDUCED COENZYME Q10-CONTAINING PARTICULATE COMPOSITION AND METHOD FOR PRODUCING THE SAME

(75) Inventors: Shinsuke Akao, Takasago-shi (JP); Takashi Ueda, Takasago-shi (JP); Takahiro Ueda, Takasago-shi (JP); Yozo Nagira, Takasago-shi (JP); Hideyuki Kishida, Takasago-shi (JP)

Correspondence Address: SUGHRUE MION, PLLC, 2100 PENNSYLVANIA AVENUE, N.W., SUITE 800, WASHINGTON, DC, 20037, US

(73) Assignee: KANEKA CORPORATION, Osaka (JP)

(21) Appl. No.: 12/579,731

(22) Filed: Oct. 15, 2009

Related U.S. Application Data
(63) Continuation-in-part of application No. PCT/JP2008/005733, filed on Apr. 15, 2008.

(30) Foreign Application Priority Data
Apr. 16, 2007 (JP) JP 2007-107202
Dec. 21, 2007 (JP) JP 2007-330191

(51) Int. Cl. A61K 38/43; A61K 31/122; A23L 1/30; A23B 4/03

(52) U.S. Cl. 424/484; 424/94.1; 514/690; 426/648; 426/465

(57) ABSTRACT

The present invention aims to propose a particulate composition containing reduced coenzyme Q10, which simultaneously shows high oxidative stability and high absorbability in the body and a production method thereof, as well as a stabilizing method thereof, to be used in the fields of foods, foods with nutrient function claims, foods for specified health uses, nutritional supplements, nutritional products, animal drugs, beverages, feeds, cosmetics, pharmaceuticals, therapeutic drugs, prophylactic drugs and the like. The present inventors have conducted intensive studies in an attempt to solve the aforementioned problems and found that a particulate composition containing reduced coenzyme Q10, wherein an oil component containing the reduced coenzyme Q10 is polydispersed forming a domain in a matrix containing a water-soluble excipient as a main component and a water-soluble ascorbic acid is a composition simultaneously having high oxidative stability and high absorbability in the body, and completed the present invention.

### TECHNICAL FIELD

The present invention relates to a particulate composition containing reduced coenzyme Q10 and a production method thereof. More particularly, the present invention relates to a particulate composition containing reduced coenzyme Q10, which simultaneously shows high oxidative stability and high oral absorbability, a production method thereof, and a dosage form that realizes high oxidative stability and high oral absorbability.

### BACKGROUND ART

Coenzyme Q is an essential component widely distributed in living organisms from bacteria to mammals. It is known that human coenzyme Q is mainly composed of coenzyme Q10, having 10 repeat structures in its side chain. Coenzyme Q10 is a physiological component present as a constituent component of the mitochondrial electron transport system in the cell of the living body. It functions as a transport component in the electron transport system by repeating oxidation and reduction in the living body.

Coenzyme Q10 is known to show energy production, membrane stabilization and antioxidant activity in the living body, and has a high degree of usability. Coenzyme Q10 occurs in two fauns, the oxidized form and the reduced form, and it is known that, in the living body, usually about 40 to 90% of the coenzyme exists in the reduced form. Of coenzymes Q10, oxidized coenzyme Q10 (aka. ubiquinone or ubidecarenone) is widely used for pharmaceutical field as a drug for cardiac failure. Besides the pharmaceutical use, it is widely used as an agent for oral preparation and a skin preparation, or as a nutritional product or a dietary supplement, like vitamin.

On the other hand, reduced coenzyme Q10 shows higher oral absorbability than oxidized coenzyme Q10, and is a superior compound effective as food, Food with nutrient function claims, Food for specified health uses, nutritional supplement, nutritional product, animal drug, drink, feed, pet food, cosmetic, pharmaceutical product, therapeutic drug, prophylactic drug and the like.

However, reduced coenzyme Q10 is easily oxidized by molecular oxygen into oxidized coenzyme Q10, and therefore, stabilization of reduced coenzyme Q10 is an important issue when it is processed into a food, food with nutrient function claims, food for specified health use, nutritional supplement, nutritional product, animal drug, drink, feed, pet food, cosmetic, pharmaceutical product, therapeutic drug, prophylactic drug and the like, or a material or composition therefor, or during handling after processing and the like. Complete removal or blocking of oxygen during the above-mentioned handling is extremely difficult and remaining or admixed oxygen particularly during heating for processing and long-term preservation exerts a markedly adverse effect. The above-mentioned oxidation is directly related to quality problems such as the by-product oxidized coenzyme Q10.

As mentioned above, stable retention (protection from oxidation) of reduced coenzyme Q10 is an extremely important problem, for which little study has been done as to the method and composition for stably retaining reduced coenzyme Q10. There are only a report on a composition concurrently containing a reducing agent and a production method thereof (patent reference 1: WO01/52822), a stabilization method in the co-presence of a reducing agent, a preservation method thereof and a composition thereof (patent document 2: WO03/32967)) and a report on stabilization of reduced coenzyme Q10 in fat and oil (patent reference 3: WO03/62182).

Patent reference 1 discloses

1) a composition comprising reduced coenzyme Q10 and an amount of a reducing agent effective to prevent the oxidation of reduced coenzyme Q10 to oxidized coenzyme Q10; and an amount of a surfactant or a vegetable oil or a mixture thereof, and optionally, a solvent effective to solubilize the above-mentioned reduced coenzyme Q10 and the aforementioned reducing agent,
2) a composition for oral administration obtained by formulating the above-mentioned composition into a gelatin capsule or tablet,
3) a method for preparing the above-mentioned composition containing reduced coenzyme Q10 by the use of oxidized coenzyme Q10 and a reducing agent in situ.

However, the above-mentioned patent reference 1 does not contain a detailed description relating to the quality, stabilizing effect and the like of reduced coenzyme Q10 contained in the composition. In the composition and preparation method thereof in patent reference 1, moreover, a reaction site for reduction of oxidized coenzyme Q10 into reduced coenzyme Q10 is provided. To do so, liposoluble oxidized coenzyme Q10 and reduced coenzyme Q10 need to be homogeneously dissolved in the same phase as a reducing agent at a molecule level, and therefore, use of a liposoluble reducing agent is in fact considered to be indispensable. In addition, patent document 2 discloses a stabilization method, a preservation method and the above-mentioned composition of reduced coenzyme Q10, which include co-presence of reduced coenzyme Q10 and ascorbic acid in the presence of a monovalent or divalent alcohol and/or a water-soluble solvent other than alcohol. However, the composition described in patent document 2 is a liquid composition and, as shown in the detailed description of the invention, the amount of the solvent to be used is preferably not less than 60 wt % of the whole mixture. Therefore, the stability of reduced coenzyme Q10 can be said to depend on the dissolution property of reduced coenzyme Q10 and ascorbic acids in a solvent.

In addition, patent reference 3 discloses, as a method for protecting reduced coenzyme Q10 from oxidation, a stabilization method of reduced coenzyme Q10, comprising forming a composition containing reduced coenzyme Q10, fats and oils (excluding olive oil) and/or polyol as a main component, which does not substantially inhibit stabilization of reduced coenzyme Q10. However, the aforementioned stabilization method may be insufficient to ensure stability of reduced coenzyme Q10. While the reference teaches that ascorbic acid may be added to the above-mentioned composition, the form of the composition reveals that only a liquid composition can be added, like the above-mentioned patent document 2.

There are several other prior art references that disclose possible addition of an antioxidant such as ascorbic acid and the like to a composition containing reduced coenzyme Q10 (e.g., patent document 4: WO05/097091). However, since reduced coenzyme Q10 is liposoluble, a liposoluble antioxidant should be used or a water-soluble antioxidant can be used only in a solvent (for example, ethanol) that can dissolve the both.

As mentioned above, for stabilization of reduced coenzyme Q10 using conventional reducing agents such as ascorbic acids and the like, or reduction of oxidized coenzyme Q10 to give reduced coenzyme Q10, a reaction to reduce oxidized coenzyme Q10 to reduced coenzyme Q10 should be ensured by

1) dissolving liposoluble oxidized and/or reduced coenzyme Q10 as homogeneously as possible in liposoluble ascorbic acids, fats and oils, and/or surfactants and an oil component of a liposoluble solvent where necessary, to achieve molecular level compatibility of liposoluble oxidized coenzyme Q10 and/or reduced coenzyme Q10 and a liposoluble reducing agent, or
2) using a large amount of a solvent (for example, ethanol etc.) that dissolves both oxidized coenzyme Q10 and/or reduced coenzyme Q10 and a reducing agent to dissolve oxidized coenzyme Q10 and/or reduced coenzyme Q10 and the reducing agent at a molecular level. In other words, the above-mentioned composition is inevitably a liquid composition whose applicable range is limited.

Under the circumstances, there is a demand for a composition containing particulate reduced coenzyme Q10 stable to oxidation, which can be used for various applications.

patent document 1: WO01/052822
patent document 2: WO03/32967
patent document 3: WO03/062182
patent document 4: WO05/097091

### DISCLOSURE OF THE INVENTION

### Problems to be Solved by the Invention

To solve the above-described problems, the present invention is directed to propose a particulate composition comprising reduced coenzyme Q10, which simultaneously shows high oxidative stability and high oral absorbability and a production method thereof, as well as a dosage form that realizes high oxidative stability and high oral absorbability, to be used in the fields of foods, foods with nutrient function claims, foods for specified health uses, nutritional supplements, nutritional products, animal drugs, beverages, feeds, pet foods, cosmetics, pharmaceuticals, therapeutic drugs, prophylactic drugs and the like.

### Means of Solving the Problems

The present inventors have conducted intensive studies in an attempt to solve the aforementioned problems and found that a particulate composition wherein an oil component containing reduced coenzyme Q10 is polydispersed forming a domain in a matrix containing a water-soluble ascorbic acids and a water-soluble excipient is a composition simultaneously having high oxidative stability and high oral absorbability, a method for producing such a particulate composition, and a dosage form thereof, which resulted in the completion of the present invention.

Accordingly, the present invention provides the following.

[1] A particulate composition comprising an oil component (A) comprising reduced coenzyme Q10, and a matrix comprising a water-soluble excipient and a water-soluble ascorbic acid, wherein the oil component (A) is polydispersed forming a domain in the matrix.
[2] The particulate composition of [1], wherein the water-soluble ascorbic acid is at least one kind selected from the group consisting of ascorbic acid, rhamno-ascorbic acid, arabo-ascorbic acid, gluco-ascorbic acid, fuco-ascorbic acid, glucohepto-ascorbic acid, xylo-ascorbic acid, galacto-ascorbic acid, gulo-ascorbic acid, allo-ascorbic acid, erythro-ascorbic acid, 6-desoxyascorbic acid and a salt thereof.
[3] The particulate composition of [1] or [2], which has a sphericity of not less than 0.8.
[4] The particulate composition of any of [1] to [3], wherein not less than 10 wt % of the reduced coenzyme Q10 in the particulate composition is non-crystalline.
[5] The particulate composition of any of [1] to [4], wherein the oil component (A) is polydispersed forming not less than 5 domains.
[6] The particulate composition of any of [1] to [5], wherein the reduced coenzyme Q10 and the water-soluble ascorbic acid are contained in the particulate composition at a weight ratio of 100:1-1:5.
[7] The particulate composition of any of [1] to [6], wherein the water-soluble excipient is at least one kind selected from the group consisting of a water-soluble polymer, surfactant (C), sugar and a yeast cell wall.
[8] The particulate composition of [7], wherein the water-soluble polymer is at least one kind selected from the group consisting of gum arabic, gelatin, agar, starch, pectin, carageenan, casein, dried albumen, curdlan, alginic acids, soybean polysaccharide, pullulan, celluloses, xanthan gum, carmellose salt and polyvinylpyrrolidone.
[9] The particulate composition of [7], wherein the surfactant (C) is at least one kind selected from the group consisting of glycerol fatty acid ester, sucrose fatty acid ester, sorbitan fatty acid ester, polyoxyethylenesorbitan fatty acid ester, lecithins and saponins.
[10] The particulate composition of [7], wherein the sugar is at least one kind selected from the group consisting of monosaccharide, disaccharide, oligosaccharide, sugar alcohol and polysaccharide.
[11] The particulate composition of any of [1] to [10], wherein the oil component (A) comprises 5-100 wt % of coenzyme Q10, 0-95 wt % of fat and oil, and 0-95 wt % of surfactant (D).
[12] The particulate composition of [11], wherein the surfactant (D) is at least one kind selected from the group consisting of glycerol fatty acid ester, polyglycerol ester, sucrose fatty acid ester, sorbitan fatty acid ester, polyoxyethylenesorbitan fatty acid ester and propylene glycol fatty acid ester, each having an HLB of not more than 10, and lecithins.
[13] The particulate composition of any of [1] to [12], wherein the content of the reduced coenzyme Q10 in the particulate composition is 1-70 wt %.
[14] The particulate composition of any of [1] to [13], wherein the volume average particle size is 1-1000 μm.
[15] The particulate composition of any of [1] to [14], wherein the domain formed by the oil component (A) has an average particle size of 0.01-50 μm.
[16] The particulate composition of any of [1] to [15], which has a residual ratio of the reduced coenzyme Q10 of not less than 80 wt % after preservation at 40° C. in the air in light shading for 30 days.
[17] A preparation obtained by processing a particulate composition of any of [1] to [16].
[18] A method of stabilizing a particulate composition or preparation comprising reduced coenzyme Q10, which comprises to placing a particulate composition of any of [1] to [16] or a preparation of [17] in an environment with surrounding relative humidity of 90% or below.
[19] A method of stabilizing a particulate composition or preparation comprising reduced coenzyme Q10, which comprises packing a particulate composition of any of [1] to [16] or a preparation of [17] with a glass, plastic and/or metal material.
[20] The method of [18] or [19] comprising concurrent use of a moisture-proof agent.
[21] A method of producing a particulate composition comprising reduced coenzyme Q10, which comprises preparing an oil-in-water emulsion composition from an oil component (a) containing coenzyme Q10 and an aqueous solution comprising water-soluble ascorbic acid and a water-soluble excipient, and then removing water in the oil-in-water emulsion composition.
[22] The method of [21], wherein the coenzyme Q10 contained in the oil component (a) is reduced coenzyme Q10.
[23] The method of [21], wherein oxidized coenzyme Q10 or a mixture of oxidized coenzyme Q10 and reduced coenzyme Q10 is used as the coenzyme Q10 to be contained in the oil component (a), and at least a part of oxidized coenzyme Q10 is reduced into reduced coenzyme Q10, during the production process of the particulate composition.
[24] The method of any of [21] to [23], wherein the water-soluble ascorbic acid is at least one kind selected from the group consisting of ascorbic acid, rhamno-ascorbic acid, arabo-ascorbic acid, gluco-ascorbic acid, fuco-ascorbic acid, glucohepto-ascorbic acid, xylo-ascorbic acid, galacto-ascorbic acid, gulo-ascorbic acid, allo-ascorbic acid, erythro-ascorbic acid, 6-desoxyascorbic acid and a salt thereof.
[25] The production method of any of [21] to [24], wherein the oil-in-water emulsified composition is suspended in the oil component (B), and thereafter the water in the emulsified composition is removed in the oil component (B).
[26] The production method of [25], wherein the oil component (B) comprises 5-99.99 wt % of fat and oil and 0.01-95 wt % of surfactant (E).
[27] The production method of [26], wherein the surfactant (E) is at least one kind selected from the group consisting of glycerol fatty acid ester, polyglycerol ester, sucrose fatty acid ester, sorbitan fatty acid ester and polyoxyethylenesorbitan fatty acid ester, each having an HLB of not more than 10, and lecithins.
[28] The method of any of [21] to [24], wherein the water in the oil-in-water emulsion composition is removed by spray drying the oil-in-water emulsion composition in a gaseous phase.
[29] The production method of any of [21] to [28], wherein the obtained particulate composition has a sphericity of not less than 0.8
[30] The production method of any of [21] to [29], wherein 1-500 parts by weight of water-soluble ascorbic acid is used relative to 100 parts by weight of coenzyme Q10.
[31] The production method of any of [21] to [30], wherein the water-soluble excipient is at least one kind selected from the group consisting of a water-soluble polymer, surfactant (C), sugar and a yeast cell wall.
[32] The production method of [31], wherein the water-soluble polymer is at least one kind selected from the group consisting of gum arabic, gelatin, agar, starch, pectin, carageenan, casein, dried albumen, curdlan, alginic acids, soybean polysaccharide, pullulan, celluloses, xanthan gum, carmellose salt and polyvinylpyrrolidone.
[33] The production method of [31], wherein the surfactant (C) is at least one kind selected from the group consisting of glycerol fatty acid ester, sucrose fatty acid ester, sorbitan fatty acid ester, polyoxyethylenesorbitan fatty acid ester, lecithins and saponins.
[34] The production method of [31], wherein the sugar is at least one kind selected from the group consisting of monosaccharide, disaccharide, oligosaccharide, sugar alcohol and polysaccharide.
[35] The production method of any of [21] to [34], wherein the oil component (a) comprises 5-100 wt % of coenzyme Q10, 0-95 wt % of fat and oil, and 0-95 wt % of surfactant (D).
[36] The production method of [35], wherein the surfactant (D) is at least one kind selected from the group consisting of glycerol fatty acid ester, polyglycerol ester, sucrose fatty acid ester, sorbitan fatty acid ester, polyoxyethylenesorbitan fatty acid ester and propylene glycol fatty acid ester, each having an HLB of not more than 10, and lecithins.

### EFFECT OF THE INVENTION

The present invention provides a particulate composition comprising reduced coenzyme Q10, which simultaneously shows high oxidative stability and high oral absorbability, and a production method thereof, and a dosage form that realizes high oxidative stability and high oral absorbability. The particulate composition comprising reduced coenzyme Q10 of the present invention, a preparation obtained by processing the composition, and the like are particularly superior in the oxidative stability under high humidity.

### BRIEF DESCRIPTION OF THE DRAWINGS

FIG. 1 A conceptional drawing of a soft capsule preparation obtained by filling a slurry mixture obtained by suspending the particulate composition of the present invention in oil component (F).

FIG. 2 A scanning electron microscopic photograph of the appearance of the particulate composition obtained in Example 1.

FIG. 3 A scanning electron microscopic photograph of the section of the particulate composition obtained in Example 1.

FIG. 4 Comparison of oral absorbability of the particulate composition obtained in Example 7 and reduced coenzyme Q10 obtained in Production Example 1.

### BEST MODE FOR EMBODYING THE INVENTION

The particulate composition of the present invention is first explained. In the particulate composition of the present invention, an oil component (A) containing reduced coenzyme Q10 is polydispersed forming a domain in a matrix containing a water-soluble excipient and water-soluble ascorbic acid.

The reduced coenzyme Q10 contained in the particulate composition of the present invention is represented by the following formula (1):

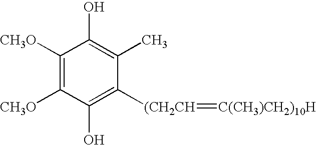

As mentioned above, coenzyme Q10 occurs in a reduced form and an oxidized form. The particulate composition of the present invention essentially contains reduced coenzyme Q10, which may be a reduced form alone or a mixture of oxidized coenzyme Q10 and reduced coenzyme Q10. When the particulate composition of the present invention contains both reduced coenzyme Q10 and oxidized coenzyme Q10, the proportion of reduced coenzyme Q10 in the total amount of coenzyme Q10 (i.e., total amount of reduced coenzyme Q10 and oxidized coenzyme Q10) is not particularly limited. For example, it is not less than about 20 wt %, generally not less than about 40 wt %, preferably not less than about 60 wt %, more preferably not less than about 80 wt %, particularly not less than about 90 wt %, and especially not less than about 96 wt %. While the upper limit is 100 wt % and is not particularly limited, it is generally not more than about 99.9 wt %.

In the present specification, a simple indication of “coenzyme Q10” means, unless otherwise specified, a mixture of oxidized coenzyme Q10 and reduced coenzyme Q10. When reduced coenzyme Q10 or oxidized coenzyme Q10 is present by itself (or used alone), the term may mean reduced coenzyme Q10 alone, or oxidized coenzyme Q10 alone.

Reduced coenzyme Q10 contained in the particulate composition of the present invention may be derived from reduced coenzyme Q10 used as a starting material for preparation of the particulate composition, or may be oxidized coenzyme Q10 used as a starting material (or a part thereof) for preparation of the particulate composition, which is then reduced in the production step to reduced coenzyme Q10.

When reduced coenzyme Q10 is used as a starting material for preparation of the particulate composition of the present invention, as described in JP-A-10-109933, for example, it can be produced by a method comprising obtaining coenzyme Q10 which is a mixture of oxidized coenzyme Q10 and reduced coenzyme Q10 by a conventionally known method such as synthesis, fermentation, extraction from a naturally occurring substance, and the like, concentrating reduced coenzyme Q10 fraction in the eluent using chromatography and the like. In this case, oxidized coenzyme Q10 contained in the above-mentioned coenzyme Q10 may be reduced with a conventional reducing agent such as sodium borohydride, sodium hydrosulfite (sodium dithionite) and the like, and concentrated by chromatography. In addition, reduced coenzyme Q10 can be obtained by reacting existing high-purity oxidized coenzyme Q10 with the above-mentioned reducing agent.

Preferably, it is obtained by reducing existing high-purity oxidized coenzyme Q10, or coenzyme Q10 which is a mixture of oxidized coenzyme Q10 and reduced coenzyme Q10, using a conventional reducing agent, for example, sodium hydrosulfite (sodium dithionite), sodium borohydride, ascorbic acids and the like. More preferably, it is obtained by reducing existing high-purity oxidized coenzyme Q10, or coenzyme Q10 which is a mixture of oxidized coenzyme Q10 and reduced coenzyme Q10, using ascorbic acids.

Water-soluble ascorbic acid contained in the matrix of the particulate composition of the present invention is not particularly limited as long as it is ascorbic acid which is soluble in water. Examples thereof include ascorbic acid, rhamo-ascorbic acid, arabo-ascorbic acid, gluco-ascorbic acid, fuco-ascorbic acid, glucohepto-ascorbic acid, xylo-ascorbic acid, galacto-ascorbic acid, gulo-ascorbic acid, allo-ascorbic acid, erythro-ascorbic acid, 6-desoxyascorbic acid and the like, as well as salts thereof. They may be L form, D form or racemates, and specifically, L-ascorbic acid, L-sodium ascorbate, L-calcium ascorbate, D-arabo-ascorbic acid and the like can be mentioned. In the present invention, the above-mentioned water-soluble ascorbic acids can be preferably used. From the aspects of stability of reduced coenzyme Q10, and broad utility, L-ascorbic acid or D-arabo-ascorbic acid is preferable. Needless to say, these water-soluble ascorbic acids may be used in combination.

The content of the above-mentioned water-soluble ascorbic acid in the particulate composition of the present invention is not particularly limited as long as it is effective for improving the oxidative stability of reduced coenzyme Q10 also contained in the particulate composition. To sufficiently improve the stability of reduced coenzyme Q10 in the obtained particulate composition, the content ratio of water-soluble ascorbic acid to 100 parts by weight of reduced coenzyme Q10 in the particulate composition is preferably 1 part by weight or above, more preferably 2 parts by weight or above, more preferably 5 parts by weight or above, particularly preferably 10 parts by weight or above.

While the upper limit of the content of water-soluble ascorbic acid in the particulate composition of the present invention is not particularly limited in view of the object of the present invention, from the economic aspects and the like, it is preferably 500 parts by weight or below, more preferably 300 parts by weight or below, more preferably 250 parts by weight or below, particularly preferably 200 parts by weight or below, per 100 parts by weight of reduced coenzyme Q10 in the particulate composition.

That is, the weight ratio of reduced coenzyme Q10 and water-soluble ascorbic acid in the particulate composition is preferably 100:1-1:5, more preferably 50:1-1:3, more preferably 20:1-1:2.5, particularly preferably 10:1-1:2.

The matrix in the present invention maintains oil component (A) containing reduced coenzyme Q10 in a particulate composition, and forms particles. The matrix in the present invention contains a water-soluble excipient and water-soluble ascorbic acid as constituent components, where a water-soluble excipient is preferably a main component, and at least water-soluble ascorbic acid is other component. The matrix may essentially consist only of a water-soluble excipient and water-soluble ascorbic acid alone.

While the content of the water-soluble excipient in the matrix component is not particularly limited, it is preferably 50 wt % or above, more preferably 55 wt % or above, more preferably 60 wt % or above, particularly preferably 80 wt % or above. The upper limit of the content of the water-soluble excipient in the matrix component is not particularly limited and, as mentioned above, the matrix component other than the water-soluble ascorbic acid may be water-soluble excipient alone. That is, the upper limit of the total amount of the water-soluble excipient and water-soluble ascorbic acid in the matrix component is 100 wt %, and when other matrix component is present, it is generally 99.9 wt % or below.

While the water-soluble excipient to be the component of the matrix in the particulate composition of the present invention is not particularly limited, it is preferably one kind selected from the group consisting of water-soluble polymer, surfactant (C), sugar and yeast cell wall, or a mixture thereof. While the above-mentioned water-soluble excipient is not particularly limited as long as it is acceptable for food, cosmetic or pharmaceutical product, one acceptable for food is particularly preferable.

As the above-mentioned water-soluble polymer, for example, gum arabic, gelatin, agar, starch, pectin, carageenan, casein, casein compound, dried albumen, curdlan, alginic acids, soybean polysaccharides, pullulan, celluloses, xanthan gum, carmellose salt (carmellose sodium, carmellose calcium and the like), higher fatty acid sugar ester, tragacanth, water-soluble polymer containing amino acid and/or sugar and the like as main components such as milk and the like, polyvinylpyrrolidone and the like can be used singly or in a mixture of two or more kinds thereof.

Of these, gum arabic, gelatin, agar, starch, pectin, carageenan, casein, dried albumen, curdlan, alginic acids, soybean polysaccharides, pullulan, celluloses, xanthan gum, carmellose salt and polyvinylpyrrolidone are preferable. Gum arabic, gelatin and soybean polysaccharides are more preferably used in view of the handlability of aqueous solution during production, or since a particulate composition simultaneously having high oxidative stability and high absorbability in the living body, which is the object of the present invention, can be obtained.

While the above-mentioned surfactant (C) is not particularly limited as long as it is acceptable for food, cosmetic and pharmaceutical product, one particularly acceptable for food is preferable. For example, glycerol fatty acid esters, sucrose fatty acid esters, sorbitan fatty acid esters, polyoxyethylenesorbitan fatty acid ester, lecithins and saponins can be used. It is needless to say that they can be used alone or in a mixture of two or more kinds thereof in the present invention.

As the aforementioned glycerol fatty acid esters, for example, fatty acid and organic acid esters of monoglycerol, polyglycerol fatty acid esters, polyglycerin condensed ricinoleate and the like can be mentioned. As the fatty acid and organic acid esters of monoglycerol, for example, stearic acid and citric acid ester of monoglycerol, stearic acid and acetic acid ester of monoglycerol, stearic acid and succinic acid ester of monoglycerol, caprylic acid and succinic acid ester of monoglycerol, stearic acid and lactic acid ester of monoglycerol, stearic acid and diacetyltartaric acid ester of monoglycerol and the like can be mentioned. As the polyglycerol fatty acid ester, for example, one having an average degree of polymerization of polyglycerin of 2-10, wherein the constituent fatty acid has 6 to 22, preferably 6-18 carbon atoms, can be mentioned. As the aforementioned polyglycerin condensed ricinoleate, for example, one having an average degree of polymerization of polyglycerin of 2-10, wherein the average degree of condensation of polyricinoleic acid (average number of condensation of ricinoleic acid) is 2 to 4, can be mentioned.

As the aforementioned sucrose fatty acid esters, one wherein one or more hydroxyl groups of sucrose is/are each esterified with fatty acid having 6 to 22, preferably 6 to 18, carbon atoms can be mentioned.

As the aforementioned sorbitan fatty acid esters, one wherein one or more hydroxyl groups of sorbitan is/are each esterified with fatty acid having 6 to 22, preferably 6 to 18, carbon atoms can be mentioned.

As the aforementioned polyoxyethylenesorbitan fatty acid esters, one wherein one or more hydroxyl groups of sorbitan is/are substituted by a polyoxyethylene chain and one or more hydroxyl groups is/are esterified with fatty acid having 6 to 22, preferably 6 to 18, carbon atoms can be mentioned.

As the aforementioned lecithins, for example, egg-yolk lecithin, purified soybean lecithin, phosphatidylcholine, phosphatidylethanolamine, phosphatidylserine, sphingomyelin, dicetyl phosphate, stearylamine, phosphatidylglycerol, phosphatidic acid, phosphatidylinositolamine, cardiolipin, ceramide phosphorylethanolamine, ceramide phosphoryl glycerol, enzymatically decomposed lecithin (lysolecithin) and a mixture thereof and the like can be mentioned.

As the aforementioned saponins, for example, enju saponin, quillaja saponin, soybean saponin, yucca saponin and the like can be mentioned.

Of the above-mentioned surfactant (C), surfactant (C) is preferably a hydrophilic surfactant and, for example, a surfactant having an HLB of not less than 4, generally not less than 6, preferably not less than 8 can be used because an oil component containing reduced coenzyme Q10 can be emulsified stably, and a particulate composition simultaneously having high oxidative stability and high absorbability in the living body, which is the object of the present invention, can be obtained.

As such surfactant, specifically, fatty acid and organic acid esters of monoglycerol such as stearic acid and citric acid ester of monoglycerol, stearic acid and diacetyltartaric acid ester of monoglycerol and the like; polyglycerol fatty acid esters such as triglycerol monolaurate, triglycerol monomyristate, triglycerol monooleate, triglycerol monostearate, pentaglycerol monomyristate, pentaglycerol trimyristate, pentaglycerol monooleate, pentaglycerol trioleate, pentaglycerol monostearate, pentaglycerol tristearate, hexaglycerol monocaprylate, hexaglycerol dicaprylate, hexaglycerol monolaurate, hexaglycerol monomyristate, hexaglycerol monooleate, hexaglycerol monostearate, decaglycerol monolaurate, decaglycerol monomyristate, decaglycerol monooleate, decaglycerol monopalmitate, decaglycerol monostearate, decaglycerol distearate and the like; polyglycerin condensed ricinoleate such as tetraglycerol condensed ricinoleate, pentaglycerol condensed ricinoleate, hexaglycerol condensed ricinoleate, diglycerol condensed ricinoleate and the like; sorbitan fatty acid esters such as sorbitan monostearate, sorbitan monooleate and the like; sucrose fatty acid esters such as sucrose palmitate, sucrose stearate and the like; lecithins such as soybean lecithin, egg-yolk lecithin, enzymatically decomposed lecithin and the like; and saponins such as enju saponin, quillaja saponin, soybean saponin, yucca saponin and the like can be mentioned.

In the present invention, surfactant (C) is preferably used in combination with other water-soluble excipient.

The above-mentioned sugar is not particularly limited as long as it is acceptable for food and, for example, monosaccharides such as glucose, fructose, galactose, arabinose, xylose, mannose and the like; disaccharides such as maltose, sucrose, lactose and the like; oligosaccharides such as fructooligosaccharide, soybean oligosaccharide, galactooligosaccharide, xylo-oligosaccharide and the like; sugar alcohols such as sorbitol, maltitol, erythritol, lactitol, xylitol and the like; polysaccharides such as dextrin and the like; and the like can be preferably used.

The dextrin is not particularly limited, and a degradation product of starch can be used, where both low molecular weight dextrin and high molecular weight dextrin can be preferably used. However, from the aspect of solubility in aqueous layer and the like, dextrin having a dextrose equivalent of generally not more than 40, preferably not more than 35, more preferably not more than 30, and generally not less than 1, preferably not less than 2, more preferably not less than 5, can be preferably used. Moreover, dextrin may be maltodextrin, cyclodextrin, cluster dextrin and the like.

As the above-mentioned yeast cell wall, beer yeast cell wall and the like can be mentioned.

In the present invention, water-soluble polymer and sugar are preferably used in combination as the water-soluble excipient. It is more preferable to combine gum arabic as the water-soluble polymer and sucrose or dextrin as the sugar. When a water-soluble polymer and sugar are used in combination, the weight ratio of water-soluble polymer and sugar is not particularly limited. The proportion of the water-soluble polymer relative to the total weight of water-soluble polymer and sugar is generally not less than 25 wt %, preferably not less than 40 wt %, more preferably not less than 50 wt %, particularly preferably not less than 60 wt %, and generally not more than 99 wt %, preferably not more than 95 wt %, more preferably not more than 90 wt %, particularly preferably not more than 85 wt %.

The oil component (A) containing reduced coenzyme Q10, which forms the domain portion of the particulate composition of the present invention may be (1) reduced coenzyme Q10 alone, or coenzyme Q10 which is a mixture of reduced coenzyme Q10 and oxidized coenzyme Q10 alone, or (2) a mixture of reduced coenzyme Q10 or coenzyme Q10, and fat and oil and/or a surfactant (D). When the oil component (A) is a mixture of reduced coenzyme Q10 or coenzyme Q10, and fat and oil and/or a surfactant (D), it is preferably an oil component that is visually uniformly mixed when heat-melted at 50° C. or above. From the aspect of maintaining a high content of reduced coenzyme Q10 in oil component (A), the above-mentioned (1) is preferable.

The fats and oils to be used when oil component (A) is the aforementioned (2) are not particularly limited and, for example, may be natural fats and oils from plants and animals, synthetic fats and oils or processed fats and oils. More preferably, one acceptable for food, cosmetic or pharmaceutical agent is used. Examples of vegetable oil include coconut oil, palm oil, palm kernel oil, flaxseed oil, camellia oil, brown rice germ oil, canola oil, rice oil, peanuts oil, corn oil, wheat germ oil, soy bean oil, perilla oil, cottonseed oil, sunflower kerel oil, kapok oil, evening primrose oil, shea butter, sal butter, cacao butter, sesame oil, safflower oil, olive oil and the like, and examples of animal fats and oils include lard, milk fat, fish oil, beef fat and the like. Furthermore, fats and oils obtained by processing them such as by fractionation, hydrogenation, transesterification (e.g., hydrogenated oil) and the like are also included. It is needless to say that medium-chain triglyceride (MCT) and the like can also be used. A mixture thereof may be used. As the medium-chain triglyceride, for example, triglyceride wherein fatty acid has 6 to 12, preferably 8 to 12, carbon atoms can be mentioned.

Of the above-mentioned fats and oils, vegetable fats and oils, synthetic fats and oils and processed fats and oils are preferable from the aspects of handlability, odor and the like. For example, coconut oil, palm oil, palm kernel oil, canola oil, rice oil, soy bean oil, cottonseed oil, safflower oil, olive oil, MCT and the like can be mentioned.

As the surfactant (D) to be used when oil component (A) is the aforementioned (2), for example, glycerol fatty acid esters, polyglycerol esters, sucrose fatty acid esters, sorbitan fatty acid esters, polyoxyethylenesorbitan fatty acid esters, propylene glycol fatty acid esters, lecithins and the like are preferable, but the surfactant is not limited to these.

As such glycerol fatty acid esters, for example, monoglycerides and diglycerides wherein fatty acid has 6 to 22, preferably 6 to 18, carbon atoms can be mentioned.

As the polyglycerol esters, for example, polyglycerin comprising polyglycerin having a polymerization degree of 2 to 10 as a main component, wherein one or more hydroxyl groups of polyglycerin is/are esterified with fatty acid having 6 to 22, preferably 6 to 18, carbon atoms can be mentioned.

As the sucrose fatty acid esters, one wherein one or more hydroxyl groups of sucrose is/are esterified with fatty acid having 6 to 22, preferably 6 to 18, carbon atoms can be mentioned.

As the sorbitan fatty acid esters, one wherein one or more hydroxyl groups of sorbitan is/are esterified with fatty acid having 6 to 22, preferably 6 to 18, carbon atoms can be mentioned.

As the polyoxyethylenesorbitan fatty acid esters, one wherein one or more hydroxyl groups of sorbitan is/are substituted by a polyoxyethylene chain and one or more hydroxyl groups is/are esterified with fatty acid having 6 to 22, preferably 6 to 18, carbon atoms can be mentioned.

As the propylene glycol fatty acid esters, for example, monoglycerides and diglycerides wherein fatty acid has 6 to 22, preferably 6 to 18, carbon atoms can be mentioned.

As the lecithins, for example, egg-yolk lecithin, purified soybean lecithin, phosphatidylcholine, phosphatidylethanolamine, phosphatidylserine, sphingomyelin, dicetyl phosphate, stearylamine, phosphatidylglycerol, phosphatidic acid, phosphatidylinositolamine, cardiolipin, ceramide phosphorylethanolamine, ceramide phosphoryl glycerol, enzymatically decomposed lecithin (lysolecithin), and a mixture thereof and the like can be mentioned.

Of the above-mentioned surfactant (D), a hydrophilic surfactant is preferable and, for example, a surfactant having an HLB of not more than 10, preferably not more than 8, more preferably not more than 6, still more preferably not more than 5 can be used because it shows good compatibility with reduced coenzyme Q10, and a particulate composition simultaneously having high oxidative stability and high absorbability in the living body, which is the object of the present invention, can be obtained. Lecithins can be preferably used without limitation by its HLB.

As such surfactant, specifically, monoglycerol monofatty acid esters such as monoglycerol monostearate, monoglycerol monooleate, monoglycerol monomyristate, monoglycerol monocaprylate, monoglycerol monolaurate, monoglycerol monobehenate, monoglycerol monoerucate and the like;

monoglycerol difatty acid esters such as monoglycerol distearate, monoglycerol dioleate, monoglycerol dicaprylate, monoglycerol dilaurate and the like;

fatty acid and organic acid esters of monoglycerol such as stearic acid and citric acid ester of monoglycerol, stearic acid and acetic acid ester of monoglycerol, hydrogenated coconut oil and acetic acid ester of monoglycerol, stearic acid and succinic acid ester of monoglycerol, caprylic acid and succinic acid ester of monoglycerol, stearic acid and lactic acid ester of monoglycerol, stearic acid and diacetyltartaric acid ester of monoglycerol and the like;

monoglycerol fatty acid esters obtained using various fats and oils such as hydrogenated beef tallow and fatty acid esters of monoglycerol, hydrogenated canola oil and fatty acid esters of monoglycerol, hydrogenated soybean oil and fatty acid esters of monoglycerol, cottonseed oil and fatty acid esters of monoglycerol, safflower oil and fatty acid esters of monoglycerol and the like;

polyglycerol fatty acid esters such as ester of polyglycerin having an average polymerization degree of 2-10 and fatty acid having 6 to 22, preferably 6 to 18, carbon atoms and the like and polyglycerin fatty acid esters such as polyglycerol condensed ricinoleic acid ester and the like (e.g., ester of polyglycerol having an average polymerization degree of 2-10 and polyricinoleic acid having a condensation degree of 2-4 and the like;

propylene glycol fatty acid esters such as propylene glycol monostearate, propylene glycol monooleate, and propylene glycol monolaurate and the like;

sorbitan fatty acid esters such as sorbitan distearate, sorbitan tristearate, sorbitan sesquioleate, sorbitan dioleate, and sorbitan trioleate and the like;

polyoxyethylenesorbitan fatty acid esters such as polyoxyethylenesorbitan monooleate, polyoxyethylenesorbitan monostearate and the like;

and a mixture of one or more kinds selected from lecithins such as soybean lecithin, egg-yolk lecithin, enzymatically decomposed lecithin and the like can be mentioned. Of these, preferred are glycerol fatty acid esters and/or lecithins, more preferred are monoglycerol monofatty acid esters, monoglycerol difatty acid esters, fatty acid and organic acid esters of monoglycerol (particularly fatty acid and acetic acid esters of monoglycerol, hydrogenated coconut oil and acetic acid ester of monoglycerol), polyglycerol fatty acid esters (particularly ester of polyglycerol having an average degree of polymerization of 2-10 and fatty acid having a carbon number of 6-22, preferably 6-18) and polyglycerin condensed ricinoleate (particularly ester of polyglycerin having an average degree of polymerization of 2-10 and polyricinoleic acid having a condensation degree of 2-4), and more preferred are fatty acid and organic acid esters of monoglycerol (particularly fatty acid and acetic acid esters of monoglycerol, hydrogenated coconut oil and acetic acid esters of monoglycerol). Specific examples thereof include 50% acetylated product of monoglycerol monostearate, completely acetylated product of hydrogenated coconut oil monoglycerides, soybean lecithin, egg-yolk lecithin, enzyme decomposition lecithin and the like. The above-mentioned surfactants can be used alone or a mixture of two or more kinds thereof.

Besides the above-mentioned, the oil component (A) in the present invention may contain, according to various objects, an oil-soluble component such as waxes, fatty acid and ester derivatives thereof and the like.

As the aforementioned waxes, for example, wax for food such as bees wax, vegetable wax, candelilla wax, rice bran wax, carnauba wax, snow wax and the like can be mentioned.

The aforementioned fatty acid and ester derivatives thereof include, but are not limited to, caprylic acid, capric acid, lauric acid, myristic acid, palmitic acid, stearic acid, oleic acid, behenic acid and esters thereof, for example, methyl ester thereof, ethyl ester thereof and the like.

While the composition ratio of an oil component (A) containing reduced coenzyme Q10 in the particulate composition of the present invention is not particularly limited, the content of reduced coenzyme Q10 in oil component (A) is preferably not less than 5 wt %, more preferably not less than 20 wt %, still more preferably not less than 40 wt %, particularly preferably not less than 50 wt %, especially preferably not less than 60 wt %, from the aspect of maintenance of a high reduced coenzyme Q10 content of the finally obtained particulate composition. The upper limit of the content of coenzyme Q10 in oil component (A) is of course 100 wt %, and use of fats and oils and surfactant other than reduced coenzyme Q10 as oil component (A) is not always necessary. However, when fat and oil or surfactant is used, the upper limit of the content of reduced coenzyme Q10 in oil component (A) is 99.99 wt %.

The content of fat and oil in oil component (A) is preferably not more than 95 wt %, more preferably not more than 75 wt %, still more preferably not more than 50 wt %, particularly preferably not more than 30 wt %. Use of fats and oils is not always necessary and the lower limit thereof is 0 wt % and generally not less than 0.01 wt % when it is to be used.

The content of the surfactant (D) in oil component (A) is preferably not more than 95 wt %, more preferably not more than 75 wt %, still more preferably not more than 50 wt %, particularly preferably not more than 30 wt %. Use of surfactant is not always necessary and the lower limit thereof is 0 wt % and generally not less than 0.01 wt % when it is to be used.

That is, as the composition, oil component (A) preferably contains 5-100 wt % of coenzyme Q10, 0-95 wt % of fat and oil, 0-95 wt % of surfactant (D), more preferably contains 20-100 wt % of coenzyme Q10, 0-75 wt % of fat and oil, and 0-75 wt % of surfactant (D), more preferably contains 40-100 wt % of coenzyme Q10, 0-50 wt % of fat and oil, and 0-50 wt % of surfactant (D), particularly preferably contains 50-100 wt % of coenzyme Q10, 0-50 wt % of fat and oil, and 0-50 wt % of surfactant (D), particularly preferably contains 55-100 wt % of coenzyme Q10, 0-45 wt % of fat and oil, and 0-45 wt % of surfactant (D), and most preferably contains 60-100 wt % of coenzyme Q10, 0-40 wt % of fat and oil, and 0-40 wt % of surfactant (D).

When the content of coenzyme Q10 in oil component (A) is less than 5 wt %, the content of reduced coenzyme Q10 in the particulate composition also decreases. As a result, when a predetermined amount of reduced coenzyme Q10 is orally administered, ingestion of a large amount of the particulate composition is necessary. Needless to say, coenzyme Q10 to be the component of oil component (A) may be reduced coenzyme Q10 alone or a mixture of reduced coenzyme Q10 and oxidized coenzyme Q10. When it is a mixture of reduced coenzyme Q10 and oxidized coenzyme Q10, the ratio of reduced coenzyme Q10 in coenzyme Q10 is preferably high.

While the content of reduced coenzyme Q10 in the particulate composition of the present invention is not particularly limited, it is generally 1 wt % or above, preferably 5 wt % or above, more preferably 10 wt % or above, and generally 70 wt % or below, preferably 60 wt % or below, more preferably 55 wt % or below. That is, the content of reduced coenzyme Q10 in the particulate composition of the present invention is preferably 1-70 wt %, more preferably 5-60 wt %, most preferably 10-55 wt %.

When the content of reduced coenzyme Q10 in the particulate composition is less than 1 wt %, oral administration of a predetermined amount of reduced coenzyme Q10 requires ingestion of a large amount of the particulate composition. While the upper limit of the reduced coenzyme Q10 content of the particulate composition is not particularly limited as long as it can afford the high oxidative stability, which is one of the objects of the present invention, when the content of reduced coenzyme Q10 in the particulate composition is higher than 70 wt %, the high oxidative stability tends to be difficult to maintain.

The average particle size of the domain formed by the oil component (A) containing reduced coenzyme Q10 in the particulate composition of the present invention is not particularly limited as long as the object of the present invention can be achieved. The average particle size is preferably 0.01-50 μm, more preferably 0.01-20 μm, most preferably 0.01-50 μm. When the average particle size of the domain formed by the oil component (A) is larger than 50 μm, oral absorbability of the reduced coenzyme Q10 in the particulate composition tends to decrease.

On the other hand, when the average particle size of the domain formed by the oil component (A) containing reduced coenzyme Q10 is smaller than 0.01 μm, problems sometimes occur in that excess surfactant (C) and/or surfactant (D) may be necessary so as to maintain the stability of emulsion droplets during the production process, an excess load is applied to an emulsification apparatus and the like. When the below-mentioned production method of the present invention is employed for the production, the average particle size of the domain formed by the oil component (A) containing reduced coenzyme Q10 can be set to a desired average particle size by controlling the emulsion particle size of oil component (a) when preparing an oil-in-water emulsion composition.

The average particle size of the domain formed by an oil component (A) containing reduced coenzyme Q10, which is contained in the particulate composition of the present invention, can be determined by rupturing a particulate composition into hemisphere, followed by image analysis of electron microscopic images of the broken-out section thereof.

In the particulate composition of the present invention, the number of domains of an oil component (A) containing reduced coenzyme Q10, which are dispersed in a matrix containing a water-soluble excipient as a main component and further containing water-soluble ascorbic acid, is preferably larger. The oil component (A) preferably forms not less than 5, more preferably not less than 1,000, more preferably not less than 10,000, domains in the matrix. While the upper limit is not particularly limited, it is generally about 1,000,000,000.

When the number of domains in the matrix containing a water-soluble excipient is less than 5, the content of reduced coenzyme Q10 in the finally-obtained particulate composition decreases, which unpreferably requires ingestion of a large amount of the particulate composition for oral administration of a given amount of reduced coenzyme Q10.

In the present invention, the particulate composition preferably shows a high sphericity, which is concretely not less than 0.8, more preferably not less than 0.85, most preferably not less than 0.9. When the sphericity of the particulate composition is high, the total surface area per unit weight of the particulate composition becomes small. As a result, the particulate composition is not easily subject to an oxidation reaction due to the oxygen molecules in the air assumed to proceed from the particle surface. On the other hand, when the sphericity of a particulate composition is low, the total surface area per unit weight of the particulate composition becomes high. As a result, the particulate composition is easily subject to an oxidation reaction due to the oxygen molecules in the air assumed to proceed from the particle surface, and a particulate composition having high oxidative stability, which is one of the objects of the present invention, tends to be difficult to obtain.

In other words, even when particulate compositions contain reduced coenzyme Q10 having the same composition, the oxidative stability of the reduced coenzyme Q10 having high oxidative stability in the particulate compositions greatly varies depending on the sphericity thereof. Preferable sphericity can be easily achieved by employing the below-mentioned preferable production method (1).

The sphericity of a particulate composition can be determined by photographing a target particulate composition with an electron microscope etc., and from a diameter ratio of the diameter of a circle having the same area and a smallest circumscribing circle, using an image analysis software WinROOF Ver. 3.30 and the like.

Moreover, in the particulate composition of the present invention, when the particle size is approximately the same, a composition having a smaller surface roughness (Ra) is more preferable. It is considered that the smaller the surface roughness (Ra) of a particulate composition is, the smaller becomes the total surface area per unit weight of the particulate composition, and the particulate composition is not easily subject to an oxidation reaction due to the oxygen molecules in the air assumed to proceed from the particle surface. In contrast, when the surface roughness (Ra) of a particulate composition is large, the total surface area per unit weight of the particulate composition becomes large. As a result, the particulate composition is easily subject to an oxidation reaction due to the oxygen molecules in the air assumed to proceed from the particle surface, and a particulate composition having high oxidative stability, which is one of the objects of the present invention, tends to be difficult to achieve.

The surface roughness (Ra) of a particle can be determined, for example, as arithmetic average surface roughness (Ra) defined in JIS B 0601-1994. The surface roughness here is considered to be in an opposite relationship with the above-mentioned sphericity, where the sphericity is high, the surface roughness tends to be small.

In the particulate composition of the present invention, moreover, not less than 10 wt %, preferably not less than 20 wt %, more preferably not less than 50 wt %, more preferably not less than 70 wt %, particularly preferably not less than 80 wt %, of reduced coenzyme Q10 in the composition is non-crystalline, i.e., amorphous or molten. A higher proportion of amorphous or molten reduced coenzyme Q10 in the particulate composition is more preferable. Needless to say, 100%, namely, the total amount of reduced coenzyme Q10 in the composition is most preferably non-crystalline, i.e., amorphous or molten.

In general, when preserved at not higher than the melting point, reduced coenzyme Q gradually shifts to a crystalline state. In the particulate composition obtained by the below-mentioned preferable production method, for example, not less than 10 wt % of the reduced coenzyme Q10 in the composition is not crystalline even after preservation at 25° C. in the air for 30 days after production. Reduced coenzyme Q10 is maintained in an amorphous or molten state in the particulate composition, rather than a crystalline state. Thus, reduced coenzyme Q10 in an oil component (A), which is released upon disintegration of the particulate composition by gastric juice or intestinal juice after oral administration, is assumed to maintain an amorphous or molten state.

In general, reduced coenzyme Q10 in an amorphous or molten state is more susceptible to emulsification in the stomach or intestine by surfactant ingredients co-existing in the living body or particulate composition, rather than reduced coenzyme Q10 in a crystalline state. As a result, absorption of reduced coenzyme Q10 in an amorphous or molten state from the gastrointestinal tract is more easily promoted than reduced coenzyme Q10 in a crystalline state. Consequently, the preferable particulate composition of the present invention is considered to acquire high oral absorbability, which is one of the objects thereof.

In the particulate composition of the present invention, its structure is controlled to allow an oil component (A) containing reduced coenzyme Q10 to be a polydispersion by forming a domain in the water-soluble excipient matrix. In a preferable production method to be mentioned later, for example, since a molten oil component (A) containing reduced coenzyme Q10 is enclosed in a microcapsule surrounded by a water-soluble excipient, the probability of development of the crystal nucleus of reduced coenzyme Q10 is assumed to drastically decrease, and the amorphous or molten state of particles is maintained for a long time after its formation. In other words, the structure of the particulate composition of the present invention, wherein an oil component (A) containing reduced coenzyme Q10 (A) is polydispersed to form a domain in a matrix containing a water-soluble excipient, is assumed to be extremely important for realizing high oral absorbability.

While the volume average particle size of the particulate composition of the present invention is not particularly limited as long as the object of the present invention can be achieved. In view of the easiness of recovery as a powder and the like, it is generally not less than 1 μm, preferably not less than 5 μm, more preferably not less than 10 μm, particularly preferably not less than 20 μm, especially preferably not less than 30 μm, most preferably not less than 50 μm. The upper limit of the volume average particle size is not particularly limited as long as the high stability and high absorbability of reduced coenzyme Q10, which is the object of the present invention, can be maintained. For easy processing into food, pharmaceutical product, cosmetic and the like, it is generally not more than 5000 μm, preferably not more than 3000 μm, more preferably not more than 2000 μm, particularly preferably not more than 100 μm, especially preferably not more than 800 μm, more especially preferably not more than 700 μm.

That is, the volume average particle size of the particulate composition of the present invention is generally 1-5000 μm, preferably 5-3000 μm, more preferably 10-2000 μm, still more preferably 20-1000 μm, particularly preferably 30-800 μm, especially preferably 50-700 μm. The volume average particle size can be measured using, for example, an ethanol solvent in a laser diffraction scattering type particle size distribution measurement apparatus (Microtruck MT3000II manufactured by NIKKISO CO., LTD., LA-950 manufactured by HORIBA Ltd., etc.).

In the particulate composition of the present invention, moreover, the absolute specific gravity of the water-soluble excipient component to be the main component of the matrix is preferably not less than 1.25, more preferably not less than 1.27, most preferably not less than 1.30. While the absolute specific gravity of the water-soluble excipient varies depending on the water content, it is only required to meet the standard level under general handling conditions or conditions where the water content is 3 wt % or below. A higher absolute specific gravity of the water-soluble excipient means formation of a dense packing state of the matrix in the particulate composition, which ensures stability of reduced coenzyme Q10 in the domain for a longer period.

While the method for achieving such absolute specific gravity value is not particularly limited, the below-mentioned production method of the present invention can be employed. Particularly, when gum arabic not in a gel state and the like are used as water-soluble excipient components, particularly high absolute specific gravity values can be obtained. In contrast, a particulate composition containing gelatin and the like as water-soluble excipient components, which is obtained according to a spray cooler method or liquid hardening method, generally shows a low absolute specific gravity of the water-soluble excipient.

While the upper limit value of the absolute specific gravity of the water-soluble excipient component is not particularly limited as long as the object of the present invention can be achieved, it is practically not more than about 1.50.

The absolute specific gravity can be measured by a known measurement method such as a liquid phase substitution method, a gaseous phase substitution method and the like. The absolute specific gravity value of the water-soluble excipient can be calculated from the density and content (wt %) of oil component (A) contained in the particulate composition.

The absolute specific gravity of the particulate composition as a whole of the present invention is not particularly limited since it is influenced by the contents of reduced coenzyme Q10 and water-soluble excipient in the particulate composition. It is, for example, generally 1.1 or above, preferably 1.2 or above, when reduced coenzyme Q10 is contained at about 15-30 wt % of the particulate composition.

In addition, the particulate composition of the present invention can contain various additives and active ingredients other than coenzyme Q10 usable for various objects in respective uses of food, cosmetics and pharmaceutical products according to each object.

For example, in addition to the above-mentioned compounds, excipients such as crystalline cellulose, calcium phosphate, calcium sulfate and the like, disintegrants such as calcium citrate, calcium carbonate, sodium hydrogen carbonate, crystalline cellulose, carboxymethylcellulose, tragacanth, alginic acid and the like, lubricants such as talc, magnesium stearate, polyethylene glycol, silica, hydrogenated oil and the like, pigments such as titanium oxide, foodcolor, colcothar, safflower pigment, caramel pigment, gardenia pigment, tar pigment, chlorophyll and the like, antiblocking agents such as stearic acid, talc, light anhydrous silicic acid, hydrated silicon dioxide and the like, absorption promoters such as higher alcohols, higher fatty acids and the like, solubilizing agents such as fumaric acid, succinic acid, malic acid and the like, stabilizers such as benzoic acid, sodium benzoate, ethyl parahydroxybenzoate, bees wax and the like can be used.

The active ingredient other than coenzyme Q10 is not particularly limited as long as it is acceptable to be used for food, cosmetic or pharmaceutical product and, for example, glutathione, L-cysteine, N-acetylcysteine, reduced alpha-lipoic acid, tocotrienol, vitamin E (α-tocopherol) and ester derivative thereof, erythorbic acid and ester derivative and salt thereof, vitamin A and ester derivative thereof, carotenoid, rutin, zeaxanthine, astaxanthin, lycopene, flavonoid, L-carnitine and pharmacologically acceptable salt thereof such as tartrate and fumarate thereof and the like, acetyl-L-carnitine, propionyl-L-carnitine, magnesium, zinc, selenium, manganese, riboflavin, niacinamide, curcuminoid, proanthocyanidin extracted from grape seed and pine bark, NADH (reduced nicotinamideadenine dinucleotide), NADPH (reduced nicotinamideadenine dinucleotide phosphate), resveratrol, bilberryan extract, milk thistle extract, highly unsaturated fatty acid obtained by concentration from fish oil and the like, and the like can be mentioned.

Preferably, glutathione, L-cysteine, tocotrienol, vitamin E (α-tocopherol) and ester derivative thereof, erythorbic acid and ester derivative and salt thereof, vitamin A and ester derivative thereof, carotenoid, rutin, astaxanthin, lycopene, flavonoid and L-carnitine can be mentioned. Of these, active ingredients having an antioxidant action such as carotenoid, astaxanthin, vitamin E and ester derivative thereof and the like are preferable from the aspect of stability of reduced coenzyme Q10.

Needless to say, various components recited here can also be used as a mixture of two or more kinds thereof. When such various additives and active ingredients are water-soluble, they are preferably contained in a matrix comprising a water-soluble excipient as a main component, and when they are liposoluble, they are contained in oil component (A) to be a domain, though nonlimitatively.

Generally, a liposoluble easily oxidizable active ingredient stabilized by microencapsulation using a water-soluble excipient tends to show lower oxidative stability under conditions where water-soluble excipient absorbs water, such as high humid conditions and the like (Y. Minemoto, et al., Food Sci. Technol. Res., 7, 91-93, 2001). In the particulate composition of the present invention, highly oxidative stability of reduced coenzyme Q10 can be realized even when the particulate compositon contains water. Therefore, the water content of the particulate composition of the present invention is not particularly limited and, for example, about 0.01-30 wt % of water can be contained. The water content of the particulate composition of the present invention is preferably 0.01-20 wt %, more preferably 0.01-wt %. When the particulate composition of the present invention has a water content higher than 30 wt %, drastic oxidative stability improving effect is difficult to obtain, though it does not apply when a desired oxidative stability can be achieved. On the other hand, the lower limit value of water content of the particulate composition is preferably low for oxidative stability of reduced coenzyme Q10 and is generally 0.01 wt % or above.

As mentioned above, the conventional step for improving the oxidative stability of reduced coenzyme Q10 is

1) dissolving liposoluble oxidized and/or reduced coenzyme Q10 as homogeneously as possible in an oil component of liposoluble ascorbic acids, fats and oils, and/or surfactants and a liposoluble solvent where necessary, to achieve molecular level compatibility of liposoluble oxidized coenzyme Q10 and/or reduced coenzyme Q10 and a liposoluble reducing agent, or
2) using a large amount of a solvent (for example, ethanol etc.) that dissolves both oxidized coenzyme Q10 and/or reduced coenzyme Q10 and a reducing agent to dissolve oxidized coenzyme Q10 and/or reduced coenzyme Q10 and the reducing agent at a molecular level. In the particulate composition of the present invention, it has been found that the stability of reduced coenzyme Q10 to oxidation can be drastically improved by adding water-soluble ascorbic acid to a matrix containing a water-soluble excipient as a main component, even when a component to be a solvent such as fats and oils, emulsifier, ethanol etc. is not present at all, or completely insufficient to achieve compatibility of reduced coenzyme Q10 and ascorbic acid at a molecular level. In other words, it has been found for the first time that oxidative stability of reduced coenzyme Q10 can be drastically improved even when the whole system is not compatible at a molecular level, and further, that the oxidative stability can be maintained even when the particulate composition absorbs water due to high humidity conditions and the like.

In the particulate composition of the present invention, the residual rate (%) of reduced coenzyme Q10 (ratio to initial weight of reduced coenzyme Q10) after preservation for 30 days at 40° C. in the air (tightly sealed or open system) under shading is preferably 80 wt % or above, more preferably 85 wt % or above, still more preferably 90 wt % or above, particularly preferably 95 wt % or above. While the humidity of the preservation atmosphere is not particularly limited, relative humidity is generally about 90% or below, preferably about 75% or below, more preferably about 60% or below, particularly preferably about 40% or below. However, as mentioned above, the particulate composition of the present invention containing water-soluble ascorbic acid in a matrix is less influenced in the oxidative stability by water content as compared to the absence of water-soluble ascorbic acid. As a result, even when the preservation atmosphere has high humidity and water content becomes high due to the moisture absorption, the particulate composition can maintain reduced coenzyme Q10 more stably.

Furthermore, when the particulate composition of the present invention is disintegrated, reduced coenzyme Q10 is released in an ultrafine state effective for absorption in the gastrointestinal tract, and therefore, the oral absorbability becomes fine. As shown in the below-mentioned Examples, the particulate composition of the present invention shows accelerated absorption rate as compared to general reduced coenzyme Q₁₀ powders, and is more rapidly absorbed in the body. As a result, blood coenzyme Q₁₀ concentration can be rapidly increased. In view of the above, ingestion of the particulate composition of the present invention before matches or practice is effective for athletes who consume a huge amount of energy. For general people, ingestion of the particulate composition of the present invention before playing sports enables them to enjoy sports in better shape. For those taking enteral feeding product due to various diseases, moreover, ingestion of coenzyme Q₁₀ that is absorbed from the intestine and effective for various diseases is highly useful. Also for such enteral feeding product users, ingestion of the particulate composition of the present invention as coenzyme Q₁₀, which is more rapidly absorbed, is very suitable. Hence, a method of improving absorption of coenzyme Q₁₀ in the body, which is characterized by administering the particulate composition of the present invention to a subject of administration, is also one embodiment of the present invention. Moreover, fast-acting preparation, sport drink, nutrition feeding product and the like, each containing the particulate composition of the present invention, is also one embodiment of the present invention.

Now the preferable production method of the particulate composition containing reduced coenzyme Q10 of the present invention is explained. The particulate composition of the present invention is preferably obtained by the following production method. However, if a similar particulate composition can be obtained by a different production method, the production method is not limited to the following.

The particulate composition containing reduced coenzyme Q10 of the present invention can be preferably produced by

(1) a method comprising suspending, in oil component (B), an oil-in-water emulsion composition prepared from an aqueous solution containing water-soluble ascorbic acid and a water-soluble excipient and an oil component (a) containing reduced coenzyme Q10, and removing water from the oil-in-water emulsion composition in oil component (B) (hereinafter referred to as production method (1)),
or
(2) a method comprising spray-drying, in a gaseous phase, an oil-in-water emulsion composition prepared from an aqueous solution containing water-soluble ascorbic acid and a water-soluble excipient and an oil component (a) containing reduced coenzyme Q10 (hereinafter referred to as production method (2)).

In the above-mentioned production methods (1) and (2), the aqueous solution to be the aqueous phase of the oil-in-water emulsion composition (hereinafter to be referred to as “aqueous solution of excipient”) is preferably used in the form of an aqueous solution wherein water-soluble ascorbic acid and a water-soluble excipient are dissolved in water. The concentration is free of any particular limitation but is preferably handled at a concentration at which the viscosity of aqueous solution does not exceed 10 Poise, preferably 5 Poise, more preferably 2 poise, still more preferably 1 poise, since the transferring property and the like can be ensured. Specific examples and preferable examples of the water-soluble ascorbic acid and water-soluble excipient here are the same as those recited in the above-mentioned explanation of the particulate composition.

The amount of the above-mentioned water-soluble ascorbic acid to be used for the production methods (1) and (2) of the present invention is not particularly limited as long as it is effective for improving the oxidative stability of reduced coenzyme Q10 in the obtained particulate composition. To sufficiently improve the oxidative stability of reduced coenzyme Q10 in the obtained particulate composition, the amount is preferably 1 part by weight or above, more preferably 2 parts by weight or above, more preferably 5 parts by weight or above, particularly preferably 10 parts by weight or above, per 100 parts by weight of coenzyme Q10 used for oil component (a). For reduction of oxidized coenzyme Q10 contained in oil component (a) in the production step, water-soluble ascorbic acid is preferably used in an amount necessary for reduction of oxidized coenzyme Q10, in addition to the amount necessary for improvement of the above-mentioned stability.

In this case, while the amount of the water-soluble ascorbic acid to be used varies depending on the ratio of oxidized coenzyme Q10 contained in coenzyme Q10, it is preferably 20 parts by weight or above, more preferably 30 parts by weight or above, still more preferably 40 parts by weight or above, particularly preferably 50 parts by weight or above, per 100 parts by weight of oxidized coenzyme Q10 in coenzyme Q10 to be used. For example, when coenzyme Q10 to be used exclusively consists of oxidized coenzyme Q10, the amount of the water-soluble ascorbic acid to be used relative to 100 parts by weight of oxidized coenzyme Q10 to be used for oil component (a) is preferably the above-mentioned amount.

While the upper limit of the content of water-soluble ascorbic acid to be used for the production method of the present invention is not particularly limited as long as the object of the present invention is achieved, it is preferably 500 parts by weight or below, more preferably 300 parts by weight or above, still more preferably 250 parts by weight or below, particularly preferably 200 parts by weight or below, per 100 parts by weight of coenzyme Q10 to be used for oil component (a), from the economical aspects and the like.

That is, the amount of the water-soluble ascorbic acid is preferably 1-500 parts by weight, more preferably 2-300 parts by weight, still more preferably 5-250 parts by weight, particularly preferably 10-200 parts by weight, per 100 parts by weight of coenzyme Q10 to be used for oil component (a).

In the above-mentioned production methods (1) and (2), a most convenient and preferable preparation method of the oil component (a) containing reduced coenzyme Q10 includes, but is not limited to, adding, where necessary, fat and oil and/or surfactant (D) and the like to reduced coenzyme Q10 melted at not less than 50° C., and mixing by stirring and the like. The coenzyme Q10 to be used for oil component (a) at this time may be reduced coenzyme Q10, a mixture of reduced coenzyme Q10 and oxidized coenzyme Q10, or oxidized coenzyme Q10 used alone.

In the production method of the present invention, as mentioned below, the ratio of reduced coenzyme Q10 in the obtained particulate composition can be increased by reducing oxidized coenzyme Q10 in the production step even when oxidized coenzyme Q10 is used alone, or coenzyme Q10 having a low reduced coenzyme Q10 content is used. Specific examples and preferable examples of other oil component (a) such as fats and oils, surfactant (D), as well as composition ratio of oil component (a) are the same as those recited in the above-mentioned explanation of the oil component (A) in the particulate composition.

In the production methods (1) and (2) of the present invention, the oil-in-water emulsion composition is prepared from the above-mentioned oil component (a) containing coenzyme Q10, and an aqueous solution of excipient. In the above-mentioned preparation method of the oil-in-water emulsion composition, for example, it is most convenient and preferable to add an oil component (a) containing coenzyme Q10 prepared at a temperature not less than the melting point of coenzyme Q10 to the above-mentioned aqueous solution of excipient, which was heated in advance to not less than 50° C., and finely disperse or emulsify oil component (a) to a desired average particle size using a known emulsification apparatus such as high-pressure homogenizer etc. In addition, it is possible to add a coenzyme Q10 powder, together with, where necessary, other oil component to an aqueous solution of excipient, which was heated in advance to not less than 50° C., melt reduced coenzyme Q10 with/without other oil component in an aqueous solution of an excipient, and emulsify the mixture, or directly add coenzyme Q10 powder or as a melt at not less than 50° C. and, where necessary, other oil component to an aqueous solution containing a water-soluble excipient, heat the mixture to not less than 50° C. to melt coenzyme Q10 and other oil component and emulsify the mixture. However, the method is not limited to these.

In the production method of the present invention, the preferable emulsion particle size of an oil component (a) of the above-mentioned oil-in-water emulsion composition is not particularly limited. When the average particle size of oil component (a) in the oil-in-water emulsion composition is large, the absorbability of the particulate composition may decrease. Thus, it is generally not more than 50 μm, preferably not more than 20 μm, more preferably not more than 15 μm, particularly preferably not more than 10 μm. When the average particle size of oil component (a) in the oil-in-water emulsion composition is small, problems occur in that excess water-soluble excipient is needed to maintain stability of emulsion droplet during the production process, excess load is applied to an emulsification apparatus and the like. Thus, the average particle size is generally 0.001 μm, preferably not less than 0.05 μm, more preferably not less than 0.01 μm.

Since the emulsion particle size of the oil component (a) in the oil-in-water emulsion composition and the domain particle size of the oil component (A) in the obtained particulate composition correlate to each other, the domain particle size of the obtained particulate composition can be controlled by controlling the particle size of the emulsion droplet in this step.

The above-mentioned emulsion particle size of oil component (a) in the oil-in-water emulsion composition can be measured using a commercially available dynamic light scattering particle size distribution analyzer or laser diffraction scattering type particle size distribution measurement apparatus.

In the production methods (1) and (2) of the present invention, the temperature of the step for preparing a oil-in-water emulsion composition from an oil component (a) and an aqueous solution of excipient and emulsion step is not particularly limited as long as it is not less than the temperature at which coenzyme Q10 in the oil-in-water composition is melted. Generally, it is not less than 50° C., preferably not less than 55° C., more preferably not less than 60° C. The upper limit is the boiling point of the system, which varies depending on the conditions such as pressurization and the like and the temperature cannot be defined generally. In the case of normal pressure conditions, the temperature is generally not more than 100° C., preferably not more than 90° C.

In the production method (1) of the present invention, the above-mentioned oil-in-water emulsion composition is mixed with a different oil component (B), and the oil-in-water emulsion composition is suspended in oil component (B) to a desired particle size, whereby an O/W/O emulsion can be produced. The above-mentioned mixing operation is, for example, most conveniently and preferably performed by adding a oil-in-water emulsion composition containing coenzyme Q10 to oil component (B) heated in advance to not less than 50° C. However, the method is not limited to this. The size of the particles suspended in the oil-in-water emulsion composition in oil component (B) can be adjusted by stirring, circulation of solution etc., or applying shear to the mixture. The temperature of oil component (B) during preparation of the mixture is preferably generally within the range of 50-120° C. to prevent rapid evaporation of water.

While the mixing ratio of the oil-in-water emulsion composition and oil component (B) in the production method (1) of the present invention is free of any particular limitation, the weight percentage of the oil-in-water emulsion composition in the mixture of the oil-in-water emulsion composition and oil component (B) is preferably not less than 1 wt %, more preferably not less than 10 wt %, particularly preferably not less than 15 wt %, from the aspect of production efficiency and the like. In addition, it is preferably not more than 70 wt %, particularly preferably not more than 60 wt %, particularly preferably not more than 50 wt %, from the aspect of suspendability in oil component (B) of the oil-in-water emulsion composition and the like. It is generally 1-70 wt %, preferably 10-60 wt %, more preferably 15-50 wt %. When the content of the oil-in-water emulsion composition in a mixture of an oil-in-water emulsion composition and oil component (B) is less than 1 wt %, the production efficiency unpreferably decreases. In addition, when the content of an oil-in-water emulsion composition in a mixture of an oil-in-water emulsion composition and oil component (B) is 70 wt % or above, the oil-in-water emulsion composition cannot be easily suspended in oil component (B).

In the production method (1) of the present invention, the above-mentioned O/W/O emulsion is afforded and then water is removed from the oil-in-water emulsion composition suspended in oil component (B). For removal of water from the oil-in-water emulsion composition, for example, the composition is heated to not less than 80° C., preferably not less than 100° C., under atmospheric pressure to evaporate water. Alternatively, a method including setting the temperature to a temperature not less than the boiling point of water (at the corresponding pressure), under any reduced pressure, and evaporating water and the like can be mentioned, but the method is not limited thereto. From the aspects of shortening of operation time and the like, the removal is preferably performed under any reduced pressure.

In the present invention, oil component (B) in production method (1) is a component containing fat and oil or, where necessary, surfactant (E). The oil component (B) is not particularly limited as long as it can suspend the above-mentioned oil-in-water emulsion composition and may be, for example, natural fats and oils from plants and animals, or synthetic fats and oils or processed fats and oils. More preferably, they are acceptable for food, cosmetic or pharmaceutical agent. Examples of the vegetable oil include coconut oil, palm oil, palm kernel oil, flaxseed oil, camellia oil, brown rice germ oil, canola oil, rice oil, peanuts oil, corn oil, wheat germ oil, soy bean oil, perilla oil, cottonseed oil, sunflower kerel oil, kapok oil, evening primrose oil, shea butter, sal butter, cacao butter, sesame oil, safflower oil olive oil, and the like, and examples of animal fats and oils include lard, milk fat, fish oil, beef fat and the like. Furthermore, fats and oils obtained by processing them by fractionation, hydrogenation, transesterification (e.g., hydrogenated oil) and the like are also included. It is needless to say that medium-chain triglyceride (MCT) can also be used. In addition, a mixture thereof may be used.

Examples of the medium-chain triglyceride include triglyceride wherein fatty acid has 6 to 12 carbon atoms, preferably 8 to 12 carbon atoms.

Of the above-mentioned fats and oils, vegetable fats and oils, synthetic fats and oils and processed fats and oils are preferable from the aspects of handlability, odor and the like. For example, coconut oil, palm oil, palm kernel oil, canola oil, rice oil, soy bean oil, cottonseed oil, safflower oil, olive oil, MCT and the like can be used.

In production method (1) of the present invention, oil component (B) may be fat and oil alone. To ensure dispersion stability of oil-in-water emulsion composition droplets dispersed in oil component (B), where necessary, oil component (B) can contain surfactant (E). The droplet of the oil-in-water emulsion composition gradually comes to have greater adhesiveness as the progress of drying, and particles tend to easily agglomerate with each other. However, in the co-presence of surfactant (E) in oil component (B), agglomeration of oil-in-water emulsion composition droplets with increased adhesiveness during drying is drastically reduced and, as a result, the recovery rate of particulate composition having a desired volume average particle size can preferably be improved strikingly.

While the content of surfactant (E) in oil component (B) is free of any particular limitation, the wt % of surfactant (E) relative to oil component (B) is generally not less than 0.001 wt %, preferably not less than 0.005 wt %, more preferably not less than 0.01 wt %, from the aspect of suppression of agglomeration during drying of the oil-in-water emulsion composition droplets and the like. While the upper limit is not particularly limited, it is generally not more than 95 wt %, preferably not more than 80 wt %, more preferably not more than 60 wt %, from the aspect of flowability of oil component (B), removal of surfactant (E) and the like.

The above-mentioned surfactant (E) is not particularly limited as long as it is acceptable to be used for food, cosmetic or pharmaceutical product. A surfactant acceptable for food is particularly preferable and, for example, surfactants such as glycerol fatty acid esters, polyglycerol esters, sucrose fatty acid esters, sorbitan fatty acid esters, polyoxyethylenesorbitan fatty acid ester and the like, which have an HLB of not more than 10 and lecithins, can be used. Needless to say, they may be used alone or in a mixture of two or more kinds thereof in the present invention.

Examples of glycerol fatty acid esters include monoglycerides and diglycerides wherein fatty acid has 6 to 22, preferably 6 to 18, carbon atoms.

Examples of polyglycerol esters include polyglycerin comprising polyglycerin having a polymerization degree of 2 to 10 as a main component, wherein one or more hydroxyl groups of polyglycerin is/are esterified with fatty acid having 6 to 22, preferably 6 to 18, carbon atoms.

Examples of sucrose fatty acid esters include one wherein one or more hydroxyl groups of sucrose is/are esterified with fatty acid having 6 to 22, preferably 6 to 18, carbon atoms.

Examples of sorbitan fatty acid esters include one wherein one or more hydroxyl groups of sorbitan is/are esterified with fatty acid having 6 to 22, preferably 6 to 18, carbon atoms.

Examples of the polyoxyethylenesorbitan fatty acid esters include one wherein one or more hydroxyl groups of sorbitan is/are substituted by a polyoxyethylene chain and one or more hydroxyl groups is/are esterified with fatty acid having 6 to 18, preferably 6 to 18, carbon atoms.

Examples of lecithins include egg-yolk lecithin, purified soybean lecithin, phosphatidylcholine, phosphatidylethanolamine, phosphatidylserine, sphingomyelin, dicetyl phosphate, stearylamine, phosphatidylglycerol, phosphatidic acid, phosphatidylinositolamine, cardiolipin, ceramide phosphorylethanolamine, ceramide phosphoryl glycerol, enzymatically decomposed lecithin (lysolecithin) and a mixture thereof and the like.

HLB of the above-mentioned surfactant (E) is preferably not more than 10, more preferably not more than 7, most preferably not more than 5 because agglomeration of oil-in-water emulsion composition droplets during drying can be efficiently suppressed. Lecithins can be preferably used without any limitation of HLB.

Among the above-mentioned surfactants (E), since coagulation of oil-in-water emulsion composition droplets during drying can be efficiently suppressed in production method (1) of the present invention, specific examples of such surfactant include monoglycerol monofatty acid esters such as monoglycerol monostearate, monoglycerol monooleate, monoglycerol monomyristate, monoglycerol monocaprylate, monoglycerol monolaurate, monoglycerol monobehenate, monoglycerol monoerucate and the like; monoglycerol difatty acid esters such as monoglycerol distearate, monoglycerol dioleate, monoglycerol dicaprylate, monoglycerol dilaurate and the like; fatty acid and organic acid esters of monoglycerol such as stearic acid and citric acid ester of monoglycerol, stearic acid and acetic acid ester of monoglycerol, hydrogenated coconut oil and acetic acid ester of monoglycerol, stearic acid and succinic acid ester of monoglycerol, caprylic acid and succinic acid ester of monoglycerol, stearic acid and lactic acid ester of monoglycerol, stearic acid and diacetyltartaric acid ester of monoglycerol and the like; monoglycerol fatty acid esters obtained using various fats and oils such as hydrogenated beef tallow and fatty acid esters of monoglycerol, hydrogenated canola oil and fatty acid esters of monoglycerol, hydrogenated soybean oil and fatty acid esters of monoglycerol, cottonseed oil and fatty acid esters of monoglycerol, safflower oil and fatty acid esters of monoglycerol and the like; polyglycerol fatty acid esters such as ester of polyglycerin having an average polymerization degree of 2-10 and fatty acid having 6 to 22, carbon atoms and the like and glycerin fatty acid esters such as polyglycerol condensed ricinoleic acid ester and the like (e.g., ester of polyglycerol having an average polymerization degree of 2-10 and polyricinoleic acid having a condensation degree of 2-4 and the like; propylene glycol fatty acid esters such as propylene glycol monostearate, propylene glycol monooleate, and propylene glycol monolaurate and the like; sorbitan fatty acid esters such as sorbitan distearate, sorbitan tristearate, sorbitan sesquioleate, sorbitan dioleate, and sorbitan trioleate and the like; polyoxyethylenesorbitan fatty acid esters such as polyoxyethylenesorbitan monostearate, polyoxyethylenesorbitan monooleate and the like; and a mixture of one or more kinds selected from lecithins such as soybean lecithin, egg-yolk lecithin, enzymatically decomposed lecithin and the like can be mentioned. Of these, preferred are glycerol fatty acid esters and/or a mixture of one or more kinds selected from lecithins such as soybean lecithin, egg-yolk lecithin, enzymatically decomposed lecithin and the like, more preferred are monoglycerol monofatty acid esters, monoglycerol difatty acid esters, fatty acid and organic acid esters of monoglycerol (particularly fatty acid and acetic acid esters of monoglycerol, hydrogenated coconut oil and acetic acid ester of monoglycerol), polyglycerol fatty acid esters (particularly ester of polyglycerol having an average degree of polymerization of 2-10 and fatty acid having a carbon number of 6-22, preferably 6-18) and polyglycerin condensed ricinoleate (particularly ester of polyglycerin having an average degree of polymerization of 2-10 and polyricinoleic acid having a condensation degree of 2-4) and a mixture of one or more kinds selected from lecithins such as soybean lecithin, egg-yolk lecithin, enzymatically decomposed lecithin and the like, and more preferred are fatty acid and organic acid esters of monoglycerol (particularly fatty acid and acetic acid esters of monoglycerol, hydrogenated coconut oil and acetic acid esters of monoglycerol). Specific examples thereof include 50% acetylated product of monoglycerol monostearate, completely acetylated product of hydrogenated coconut oil monoglycerides, a mixture of one or more kinds from soybean lecithin, egg-yolk lecithin, enzyme decomposition lecithin and the like.

In the production method (1) of the present invention, use of MCT as fat and oil and egg-yolk lecithin, soybean lecithin or enzymatically decomposed lecithin as surfactant (E) in combination is particularly preferable.

In the production method (1) of the present invention, the time necessary for removing water from oil-in-water emulsion composition droplets is free of any particular limitation. It is preferably within the range of 1 sec-24 hr, more preferably 3 sec-12 hr, most preferably 5 sec-6 hr. When the time necessary for removing water is less than 1 sec, violent bubbling may occur due to instantaneous evaporation of water from oil component (B). On the other hand, when the time necessary for removing water is longer than 24 hr, the productivity is degraded.

Even if water is not completely removed, removal of water in the production method (1) of the present invention is sufficient as long as drying of oil-in-water emulsion composition droplets proceeds and recovery as particles is possible. The water content of particulate composition is generally preferably not more than 30 wt %, more preferably not more than 20 wt %, most preferably not more than 15 wt %, of the weight of recovered particles. Needless to say, the lower limit is 0 wt %, but it is generally 0.01 wt % or above.

In the above-mentioned production method (1), the method of recovering the particulate composition after removal of water is not particularly limited. It is most convenient and preferable to remove oil component (B) by solid-liquid separation, wash the obtained particulate composition with an organic solvent etc. to wash away most part of oil component (B), dry the organic solvent and recover the composition as a powder.

The organic solvent used for washing oil component (B) is not particularly limited as long as it can dissolve and remove oil component (B). It is preferably an organic solvent usable for the production of food, pharmaceutical product, cosmetic and the like. Examples of the solvent include, but is not limited, ethanol, methanol, isopropanol, acetone, hexane, ethyl acetate, tetrahydrofuran and the like. Of these, ethanol is most preferable when the particulate composition of the present invention is used for food. The above-mentioned organic solvent can be dried by, but is not limited to, vacuum drying, drying by heating, air drying and the like. The particulate composition after recovery may be subjected to a classification operation to have a desirable particle size of a given product.

In the production method (2) of the present invention, as mentioned above, the particulate composition of the present invention can be obtained by spray drying, in a gaseous phase, a oil-in-water emulsion composition prepared from an oil component (a) containing coenzyme Q10 and an aqueous solution of excipient. For spray drying in a gaseous phase, what is called a spray dry method can be used. The conditions for spray drying can be appropriately selected from the conditions generally employed.

Of the above-mentioned two kinds of production methods, production method (1) is a more preferable production method since a particulate composition having high oxidative stability, high sphericity and small surface roughness (Ra), which is the object of the present invention, tends to be easily obtained because removal of water proceeds while individual oil-in-water emulsion composition droplets suspended in a nearly spherical shape in oil component (B) maintain the spherical shape.

A particulate composition containing reduced coenzyme Q10 having a nearly spherical shape and small surface roughness (Ra) can also be formed by production method (2) by appropriately controlling the temperature and residence time and the like during drying.

In the production methods (1) and (2) of the present invention, naturally, to suppress oxidation of reduced coenzyme Q10 in the production process, each operation can be performed under deoxygenation atmosphere.

In the production methods (1) and (2) of the present invention, when coenzyme Q10 containing oxidized coenzyme Q10, or oxidized coenzyme Q10 itself is used as a production starting material, the proportion of reduced coenzyme Q10 in the obtained particulate composition can also be increased by reducing at least a part of the oxidized coenzyme Q10 with water-soluble ascorbic acid used in the production process thereof. As a production step wherein oxidized coenzyme Q10 is reduced with water-soluble ascorbic acid, for example, the preparation process of the aforementioned oil-in-water emulsion composition, the process of removing water from the oil-in-water emulsion composition and the like can be mentioned. Still in these cases, for easy control of the reduced coenzyme Q10 content (or weight ratio of reduced coenzyme Q10 in coenzyme Q10) of the obtained particulate composition, the weight ratio of reduced coenzyme Q10 in coenzyme Q10 used as a starting material is preferably high.

By applying a method for increasing the proportion of reduced coenzyme Q10 in the above-mentioned particulate composition, oxidized coenzyme Q10 can be reduced to produce reduced coenzyme Q10. That is, a production method of reduced coenzyme Q10, comprising preparing an oil-in-water emulsion composition from an aqueous solution containing water-soluble ascorbic acid and a water-soluble excipient and oil component (a) containing coenzyme Q10, and reducing oxidized coenzyme Q10 of coenzyme Q10 in the oil-in-water emulsion composition is also one embodiment of the present invention (hereinafter to be referred to as “production method (3) of the present invention”).

The coenzyme Q10 that can be used in production method (3) of the present invention is not particularly limited as long as it is coenzyme Q10 containing at least oxidized coenzyme Q10, and it may be a mixture of oxidized coenzyme Q10 and reduced coenzyme Q10, or oxidized coenzyme Q10 alone. Specific examples and preferable examples of the water-soluble ascorbic acid to be used for the reduction reaction and the amount of use thereof are the same as those recited in the above-mentioned explanation of the above-mentioned particulate composition and production methods (1) and (2) of the present invention. While the water-soluble excipient that can be used for the production method (3) of the present invention is not particularly limited, it is, for example, preferably one kind selected from water-soluble polymers such as gum arabic, gelatin, agar, starch, pectin, carageenan, casein, dried albumen, curdlan, alginic acids, soybean polysaccharides, pullulan, celluloses, xanthan gum, carmellose salt and polyvinylpyrrolidone, sugar, a yeast cell wall and the like, or a mixture thereof, and surfactant (C) may also be contained. Specific examples and preferable examples of the water-soluble excipient and the amount of use thereof are the same as those recited in the above-mentioned explanation of the above-mentioned particulate composition and production methods (1) and (2) of the present invention. As the preparation method and the like of the oil-in-water type emulsion, the methods described in the above-mentioned production methods (1) and (2) of the present invention can be directly applied. While the reaction temperature is not particularly limited, it is generally preferably 20° C. or above, 40° C. or above, more preferably 50° C. or above, and particularly preferably 60° C. or above, so as to shorten the reaction time and the like. While the upper limit is the boiling point of the system, it is, for example, 100° C. or below or 90° C. or below.

The stabilization method of the above-mentioned particulate composition containing reduced coenzyme Q10 of the present invention is now explained.

The stabilization as referred to in the present specification means suppression of oxidation of reduced coenzyme Q10 to oxidized coenzyme Q10. The stabilization method in the present invention can also be called a method of stably handling a particulate composition comprising reduced coenzyme Q10 against oxidation. The handling as referred to in the present specification means maintaining or exerting the function of a certain object by applying an external action on the object.

While examples of handling are not limited, they include taking out from a coating machine, wrapping, packing, preservation, storage and transport, with preference given to preservation.

The upper limit of the temperature of the handling condition in the stabilization method of the particulate composition containing reduced coenzyme Q10 of the present invention is generally not more than about 100° C., preferably not more than about 80° C., more preferably not more than about 60° C., more preferably not more than about 40° C., particularly preferably not more than about 20° C. In this case, the lower limit of the temperature is generally not less than about −100° C., preferably not less than about −80° C., more preferably not less than about −60° C., more preferably not less than about −40° C., particularly preferably not less than about −20° C.

The present invention can further stabilize a particulate composition containing reduced coenzyme Q10 and a preparation containing the composition, which is characterized by controlling the relative humidity under the handling condition.

The particulate composition of the present invention containing water-soluble ascorbic acid in a matrix containing a water-soluble excipient as a main component can dramatically minimize the influence of humidity in handling environment as compared to a particulate composition free of water-soluble ascorbic acid. In consideration of the long-term preservation stability, however, the atmosphere conditions in the handling environment preferably show lower humidity. Generally, a particulate composition of the present invention containing reduced coenzyme Q10 can be more stably handled under an environment adjusted to relative humidity of not more than about 90%, preferably not more than about 80%, more preferably not more than about 70%, particularly preferably not more than about 60%. The lower limit of the relative humidity is 0%.

The above-mentioned environment with adjusted relative humidity can be afforded by dehumidification of the environment or introduction of a dehumidificated gas (e.g., air, preferably dry inert gas such as dry nitrogen and the like) into the environment and the like. While the above-mentioned dehumidification is not particularly limited, it is achieved by moisture freezing, use of a dehumidification machine, desiccant agent (silica gel, calcium chloride, synthesis zeolite etc.) and the like. Needless to say, the method is not particularly questioned as long as the environment with adjusted relative humidity can be afforded.

To maximally exert the effect of the invention and from the aspect of the stability of reduced coenzyme Q10, the production and preservation of the particulate composition of the present invention is naturally preferably performed under a deoxygenation atmosphere. For example, it is preferably performed under a deoxygenation atmosphere using an inert gas such as nitrogen gas, argon gas etc., and the like.

In the present invention, moreover, reduced coenzyme Q10 in the particulate composition of the present invention can be stably preserved for a long time by wrapping or packing the particulate composition with glass, plastic and/or metal material(s). The form of wrapping or packing may be tight sealing (closed) or open, with preference given to a tightly sealed wrapping or packing.

As the glass material, for example, soft glass, hard glass and the like can be used. As the plastic material, for example, high density polyethylene, medium density polyethylene, low density polyethylene, polypropylene, poly(ethylene terephthalate), polyvinyl alcohol, polyvinyl chloride, polyvinylidene chloride, nylon and the like can be used. Needless to say, a film laminated with the above-mentioned plastic material, a film laminated with aluminum and the like on a plastic material such as aluminum laminate and the like, and a film obtained by vapor depositing aluminum, alumina, silica and the like on a plastic material are also included in the plastic materials. As the metal material, for example, iron, aluminum, zinc, nickel, cobalt, copper, tin, titanium, chrome or alloy thereof (stainless, brass etc.) can be used. In addition, an enameled material using glass and metal in combination and the like can also be used.

The above-mentioned materials are preferably formed into a bottle, bag, can, drum, box and the like and used for wrapping or packaging the particulate composition of the present invention. Using the above-mentioned materials, moreover, PTP packaging, three-sided seal packaging, four-sided seal packaging, pillow packaging, strip packaging, aluminum molded packaging, stick packaging and the like can also be performed. When a material having comparatively low gas barrier and moisture-proof properties such as polyethylene and the like is used, double wrapping or packing or more is preferable. In this case, use of a material having comparatively high gas barrier and moisture-proof properties such as aluminum laminate, vapor deposition films (e.g., aluminum, alumina, silica and the like), glass, metal and the like is particularly preferable. After wrapping and packing, the composition can be transported or preserved in, where necessary, iron steel drum, resin drum, fiber drum, corrugated board and the like. In this case, a moisture-proof agent such as silica gel, calcium chloride, synthetic zeolite and the like can also be enclosed.

The residual ratio (%) of reduced coenzyme Q10 after preservation of a particulate composition comprising reduced coenzyme Q10 of the present invention at 40° C. in the air for 30 days under shading conditions in the stabilization method of the present invention is not particularly limited. It is generally not less than about 80 wt %, preferably not less than about 85 wt % and more preferably not less than about 90 wt %.

Now, a preferable dosage form of the particulate composition comprising reduced coenzyme Q10 of the present invention is explained.

The particulate composition containing reduced coenzyme Q10, which is obtained in the present invention, can be processed into or used as a pharmaceutical agent, food, cosmetic and the like in the form of a preparation such as tablet, pill, capsule (hard capsule, soft capsule, microcapsule and the like), chewable tablet, powder preparation, granule, syrup, drinkable preparation and the like, and the like, whereby a dosage form that realizes high oxidative stability and high oral absorbability, that are the objects of the present invention, can be realized. That is, the preparation in this context does not refer solely to a pharmaceutical agent but also encompasses the aforementioned form belonging to food and cosmetics. For preparation making, excipient, disintegrant, lubricant, binder, anticoagulant, absorption promoter, dissolving agent, stabilizer, antioxidant and the like can be used. For forming a capsule, fat and oil, surfactants such as lecithin, lysolecithin and the like can also be used in combination.

For example, the particulate composition of the present invention is processed into a mixed slurry by suspending the composition in an oil component (F), and the slurry is filled in a soft capsule of gelatin and the like, whereby a soft capsule preparation realizing high oxidative stability and high oral absorbability can be afforded.

Conventionally, as a soft capsule preparation containing reduced coenzyme Q10, a preparation obtained by filling, in a soft capsule, a composition wherein a reduced coenzyme Q10 powder is dispersed or dissolved, like a slurry, in an oil component containing vegetable oil and/or surfactant as a main component is known. However, in this preparation, entry of oxygen from the outside is physically blocked only by the outer skin of the capsule, in an attempt to stabilize reduced coenzyme Q10. In particular, the stability of reduced coenzyme Q10 in the capsule is not sufficient under high humidity conditions during preservation.

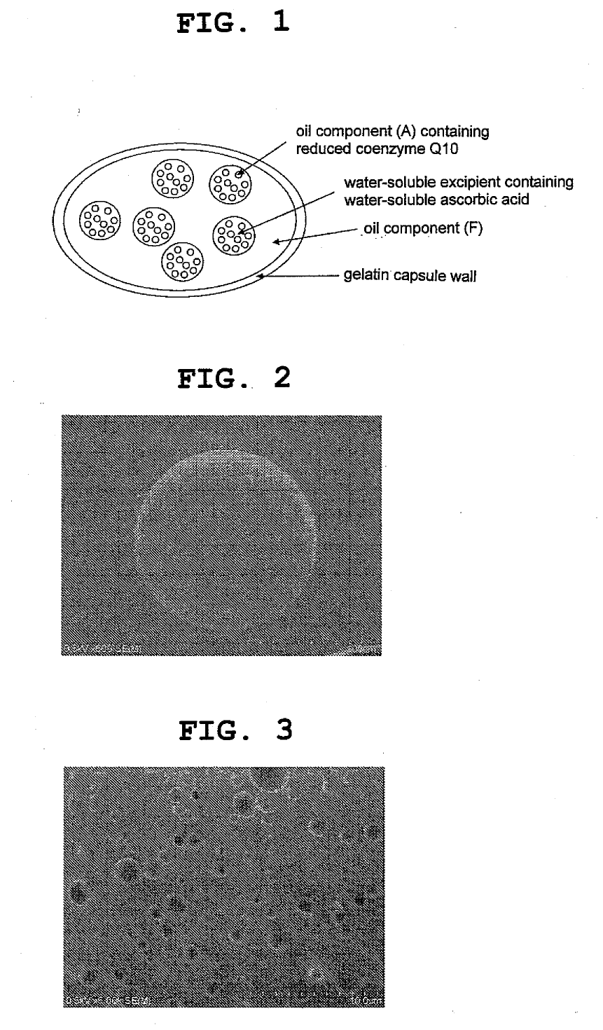

On the other hand, in a soft capsule preparation obtained by filling, in a soft capsule made of gelatin and the like, a mixed slurry obtained by suspending the particulate composition of the present invention in oil component (F), entry of oxygen from the outside can be physically blocked by double films including a water-soluble excipient layer, in addition to a capsule outer skin, and a preparation showing good oxidative stability of reduced coenzyme Q10 can be afforded. A conceptual drawing is shown in FIG. 1.

Since a soft capsule preparation obtained by processing the particulate composition of the present invention can be stably handled and/or preserved even under high humidity conditions, it is particularly superior in the stability under high humidity conditions as compared to conventional soft capsule preparations.

As the oil component (F) to be used for the above-mentioned soft capsule preparation, the aforementioned fats and oils, surfactants (emulsifiers), wax such as beeswax and the like, and the like can be used alone or a mixture of two or more kinds thereof. However, component (F) is not limited to these, and other components may be added as necessary so that the oral absorbability of reduced coenzyme Q10 in a capsule will be fine, or a combination agent with any other active ingredients may be formed. Needless to say, the above-mentioned fats and oils, surfactants and wax, which are acceptable for food, pharmaceutical product and the like, are preferable.

In the present invention, moreover, the above-mentioned particulate composition of the present invention may be directly filled in a hard capsule made of gelatin and the like, or filled as a powder obtained by mixing with any general preparation components such as excipient, lubricant and the like, or as a slurry obtained by suspending in the above-mentioned oil component (F), whereby a hard capsule preparation capable of realizing high oxidative stability and high oral absorbability can be afforded.

Generally, as a hard capsule preparation containing reduced coenzyme Q10, a preparation obtained by filling a powder composition containing a reduced coenzyme Q10 powder in a hard capsule made of gelatin and the like is assumed. In this preparation, however, entry of oxygen from the outside is physically blocked by the capsule outer skin alone, like the above-mentioned soft capsule preparation, and the reduced coenzyme Q10 thus filled may be easily oxidized even after formulation of the preparation.

In contrast, in a hard capsule preparation obtained by filling a particulate composition containing reduced coenzyme Q10 of the present invention in a hard capsule made of gelatin and the like, entry of oxygen from the outside can be physically blocked by double films including a water-soluble excipient layer, in addition to the capsule outer skin, and a reduced coenzyme Q10 preparation more stable to oxidation can be afforded.

Needless to say, any other components may be added as necessary so that the oral absorbability of reduced coenzyme Q10 of the present invention will be fine, or a combination agent with any other active ingredients may be formed.

Moreover, in the present invention, the above-mentioned particulate composition of the present invention can be processed into tablet or chewable agent together with any excipient, lubricant and the like.

Generally, as a tablet or chewable agent containing reduced coenzyme Q10, a preparation obtained by processing a composition obtained by directly mixing a reduced coenzyme Q10 powder with an excipient and the like by tableting and the like is assumed. In this preparation, however, a naked reduced coenzyme Q10 powder is only dispersed and distributed in a composition containing an excipient such as lactose and the is like, lubricant such as magnesium stearate, crystalline cellulose and the like, and the like, and reduced coenzyme Q10 cannot be kept from oxidation. As a result, the oxidative stability inevitably reaches a low level. However, in a tablet or chewable agent obtained by processing the particulate composition comprising reduced coenzyme Q10 of the present invention, since reduced coenzyme Q10 is covered with a film of a matrix of a water-soluble excipient, which contains water-soluble ascorbic acid, entry of oxygen from the outside can be physically blocked, whereby a reduced coenzyme Q10 preparation more stable to oxidation can be obtained. Needless to say, any other components may be added as necessary so that the oral absorbability of reduced coenzyme Q10 of the present invention will be fine, or a combination agent with any other active ingredients may be formed. In addition, a coating such as sugar coating and the like can be applied as necessary to the tablet or chewable agent of the present invention.

From the aspect of the stability of a particulate composition containing reduced coenzyme Q10, in a preferable embodiment of the above-mentioned preparation, handling or preservation in the aforementioned environment where the humidity has been adjusted and/or the aforementioned wrapping or packing for handling or preservation is employed.

Moreover, the particulate composition of the present invention may be directly or, after dissolution and/or dispersion in water, used for food such as jelly, yogurt and the like, drinks, cosmetics and the like, each containing reduced coenzyme Q10. In addition, the particulate composition of the present invention may be used by directly dissolving and/or dispersing in commercially available drinks, cosmetics and the like. In respective production processes of such food, drinks or cosmetics, the particulate composition of the present invention may be disintegrated in water in some cases, and the oil component containing reduced coenzyme Q10 in the domain becomes fine particles having a volume average particle size of 0.01-50 μm and is dispersed in the product. Using the particulate composition of the present invention, reduced coenzyme Q10 can be preserved with good oxidative stability until immediately before production of the product, reduced coenzyme Q10 can be microdispersed to have an extremely small particle size in the product, and a reduced coenzyme Q10-containing emulsion composition can be obtained under mild stirring conditions even without a large emulsification apparatus.

The emulsion particle size of the oil component in this case can also be measured by the aforementioned commercially available dynamic light scattering particle size distribution analyzer or laser diffraction scattering particle size distribution measurement apparatus and the like.

The particulate composition of the present invention, in the above-mentioned form or other form, can be used widely for applications as food (general foods, food with nutrient function claims, food for specified health uses, nutritional supplement, nutritional product), animal drugs, drinks, feed, pet food, cosmetic, pharmaceutical product, therapeutic drug, prophylactic drug and the like, a material thereof, a material to be processed into a composition and the like.

### EXAMPLES

The present invention is explained in more detail in the following by referring to Examples, which are not to be construed as limitative.

### (Purity of Reduced Coenzyme Q10)

The purity of reduced coenzyme Q10, the weight ratio (%) of oxidized coenzyme Q10 and reduced coenzyme Q10 and the like were determined by the following HPLC analysis. The HPLC analysis conditions are described below.

column: SYMMETRY C18 (manufactured by Waters) 250 mm (length) 4.6 mm (inner diameter),
mobile phase; C₂H₅OH/CH₃OH=4/3(v/v),
detection wavelength; 210 nm,
flow rate; 1.0 ml/min,
retention time of reduced coenzyme Q10; 9.1 min,
retention time of oxidized coenzyme Q10; 13.3 min.

### (Sphericity)

The sphericity of the obtained particulate composition was determined by analyzing, using an image analysis software (WinROOF Ver. 3.30), the images obtained by observation of the recovered particles with an electron microscope and from a diameter ratio of the diameter of a circle and a smallest circumscribing circle, both having the same area. For the analysis, 20 samples were analyzed and the average value was obtained.

### (Crystallinity)

The crystallinity of reduced coenzyme Q10 in the obtained particulate composition was determined by the following DSC (differential scanning calorimeter [EXSTAR6000 manufactured by Seiko Instruments Inc.]) analysis after preservation at 25° C. in the air for 30 days. The particulate compositions obtained in Examples and Comparative Examples were preserved under the above-mentioned given conditions, 10 mg thereof was taken in an aluminum pan and the temperature was elevated from 15° C. to 70° C. at a temperature rise rate of 5° C./min, during which the crystal melting calorie was measured. The crystallinity was calculated according to the following formula using the theoretical melting calorie determined from the content of reduced coenzyme Q10 in the particulate composition and the data of melting calorie actually measured by DSC.

Crystallinity (%)=(measured melting calorie/theoretical melting calorie)×100

### (Volume Average Particle Size)

The volume average particle size of the obtained particulate composition was measured by a laser diffraction scattering type particle size distribution measurement apparatus (Microtruck MT3000II manufactured by NIKKISO CO., LTD. or manufactured by HORIBA, Ltd.; 950) using an ethanol solvent.

### (Emulsion Particle Size)

The emulsion particle size of oil component (A) of the obtained oil-in-water emulsion composition was measured by a dynamic light scattering particle size analyzer (LB-550 manufactured by HORIBA, Ltd.).

### (Domain Average Particle Size)

The obtained particulate composition was added to a two-component curable adhesive (Araldite handled by As One Co. Ltd.) and cured. The obtained embedded sample was immersed in liquid nitrogen for 5 min, sufficiently cooled and ruptured using a hammer. The broken-out section was immersed in hexane for 15 min to remove oil component (A), and the broken-out section of the particulate composition was photographed with a scanning electron microscope (S-4800 manufactured by Hitachi High-Technologies Corporation). The average particle size of the domain was determined by selecting any 50 voids from randomly taken images, measuring the particle size thereof and taking the average thereof.

### (Measurement of Absolute Specific Gravity)

The absolute specific gravity of the obtained particulate composition was measured according to a liquid phase substitution method. The detail is shown in the following. In the liquid phase substitution method, AUTO TRUE DENSER MAT-7000 (manufactured by SEISHIN ENTERPRISE Co., Ltd.) was used and the absolute specific gravity was measured under the conditions of a dry ethanol solvent at 25±2° C.

The reduced coenzyme Q10 dissolved by heating at 60° C. was filled in a 20 cc measuring flask under an atmosphere at 60° C., and the weight of the reduced coenzyme Q10 in a liquid state was measured. The density of the reduced coenzyme Q10 in a liquid state was calculated from the obtained weight value to find 0.925 g/cm³. The obtained value was used as the representative density of the reduced coenzyme Q10 necessary for the calculation of the absolute specific gravity of the component of water-soluble excipient.

The obtained particulate composition was heated at 100° C. for 2 hr, and the water content (volatilization content) of the particulate composition was adjusted to not more than 3 wt %. Then, the absolute specific gravity of the particulate composition after the water content adjustment was measured under the above-mentioned conditions to give absolute specific gravity d of the particulate composition. The absolute specific gravity of the water-soluble excipient component was calculated from the following formula:

absolute specific gravity of water-soluble excipient component=[d−{(c/100)×0.925}]/[(100−c)/100]

wherein d shows the absolute specific gravity of the particulate composition, 0.925 shows the density of oil component (A) (0.925 g/cm³ in the case of reduced coenzyme Q10 alone), and c is the content (wt %) of oil component (A) in the particulate composition.

### Production Example

Oxidized coenzyme Q10 crystal (100 g, manufactured by Kaneka Corporation) and L-ascorbic acid (60 g) were added to ethanol (1000 g) and the mixture was stirred at 78° C. to carry out a reduction reaction. After 30 hr, the mixture was cooled to 50° C., and ethanol (400 g) and water (100 g) were added while maintaining the same temperature. With stirring, the ethanol solution was cooled to 2° C. at a cooling rate of 10° C./hr to give a white slurry. The obtained slurry was filtered under reduced pressure, wet crystals were washed with cold ethanol and cold water in this order, and the obtained wet crystals were dried under reduced pressure to give white dry crystals (95 g) (yield 95 mol %). All the operations except drying under reduced pressure were performed under a nitrogen atmosphere.

The purity of the obtained crystals was 99.1% and weight ratio of reduced coenzyme Q10/oxidized coenzyme Q10 was 99.5/0.5.

### Example 1

Gum arabic (60 g, gum arabic A manufactured by Ina Food Industry Co., Ltd.) and L(+)-ascorbic acid (1.9 g, manufactured by Wako Pure Chemical Industries, Ltd.) were dissolved in distilled water (140 g) at 60° C. to give an aqueous solution of water-soluble excipient. The aqueous solution was maintained at 60° C., the reduced coenzyme Q10 powder (9.2 g) obtained in the above-mentioned Production Example 1 was added and melted, and the mixture was emulsified by TK homomixer Mark II (manufactured by PRIMIX Corporation) at 10000 rpm×5 min to give an oil-in-water emulsion composition. The emulsion particle size of the reduced coenzyme Q10 in the oil-in-water emulsion composition was about 1 μm. The oil-in-water emulsion composition (75 g) obtained here was added to oil component (B) heated to 90° C. in advance, which comprised MCT (100 g, Actor M-2 manufactured by Riken Vitamin Co., Ltd.), and tetraglycerol pentaoleate (50 g, manufactured by Sakamoto Yakuhin Kogyo Co., Ltd.; SY Glyster PO-3S, HLB 3.0), and the number of stirring rotation was adjusted such that the oil-in-water emulsion composition would be suspended in the oil component (B). The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The sphericity of the obtained particulate composition was 0.97, volume average particle size was about 200 μm coenzyme Q content of the particulate composition was 12.6 wt %, and the weight ratio of reduced coenzyme Q10/oxidized coenzyme Q10 was 99.7/0.3. In addition, the residual ratio of reduced coenzyme Q10 after preservation of the particulate composition sealed in a glass bottle for 90 days at 40° C. under shading conditions was 97.8%. In addition, the residual ratio of reduced coenzyme Q10 after preservation for 30 days in an environment at 40° C., relative humidity 80% in the air (open) under shading conditions was 97.7%. Moreover, the crystallinity of reduced coenzyme Q10 in the particulate composition as measured by DSC was 0%, and 100 wt % of amorphous or molten reduced coenzyme Q10 was contained.

The appearance of the obtained particulate composition was observed with a scanning electron microscope (S-4800 manufactured by Hitachi High-Technologies Corporation) to find an extremely good spherical shape as shown in FIG. 2. Furthermore, the cross section of the obtained particulate composition was observed with a scanning electron microscope after dissolving and removing reduced coenzyme Q10 by immersion in hexane. As a result, as shown in FIG. 3, the matrix comprising a water-soluble excipient as a main component had a trace of multidispersion of at least 100 domains of oil component (A) containing reduced coenzyme Q10. In addition, it was also found that the average particle size of the domain of oil component (A) was about 1 μm, and the emulsion particle size of reduced coenzyme Q10 in the oil-in-water emulsion composition in the production step was maintained even after preparation of the particles.

### Comparative Example 1

Gum arabic (60 g, gum arabic A manufactured by Ina Food In dustry Co., Ltd.) was dissolved in distilled water (140 g) at 30° C. to give an aqueous solution of water-soluble excipient. The aqueous solution was heated to 60° C., the reduced coenzyme Q10 powder (9.2 g) obtained in the above-mentioned Production Example 1 was added and melted, and the mixture was emulsified by TK homomixer Mark II (manufactured by PRIMIX Corporation) at 10000 rpm×5 min to give an oil-in-water emulsion composition. The emulsion particle size of the reduced coenzyme Q10 in the oil-in-water emulsion composition was about 1 μm. The oil-in-water emulsion composition (75 g) obtained here was added to oil component B) heated to 90° C. in advance, which comprised MCT (100 g, Actor M-2 manufactured by Riken Vitamin Co., Ltd.), and tetraglycerol pentaoleate (50 g, manufactured by Sakamoto Yakuhin Kogyo Co., Ltd.; SY Glyster PO-3S, HLB 3.0), and the number of stirring rotation was adjusted to the same conditions with Example 1. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The volume average particle size of the obtained particulate composition was about 200 μm, coenzyme Q content of the particulate composition was 13.0 wt %, and the weight ratio of reduced coenzyme Q10/oxidized coenzyme Q10 was 99.2/0.8. In addition, the residual ratio of reduced coenzyme Q10 after preservation of the particulate composition sealed in a glass bottle for 90 days at 40° C. under shading conditions was 95.8%. In addition, the residual ratio of reduced coenzyme Q10 after preservation for 30 days in an environment at 40° C., relative humidity 80% in the air (open) under shading conditions was 77.6%.

### Example 2

Gum arabic (60 g, gum arabic A manufactured by Ina Food Industry Co., Ltd.) and L(+)-ascorbic acid (1.9 g, manufactured by Wako Pure Chemical Industries, Ltd.) were dissolved in distilled water (140 g) at 60° C. to give an aqueous solution of water-soluble excipient. Separately, the reduced coenzyme Q10 powder (9.2 g) obtained in the above-mentioned Production Example 1 and diglycerol monooleate (4.6 g, poem DO-100V manufactured by Riken Vitamin Co., Ltd.) were uniformly mixed at 60° C. to give oil component (a), which was added to an aqueous solution of water-soluble excipient at 60° C., and the mixture was emulsified by TK homomixer Mark II (manufactured by PRIMIX Corporation) at 10000 rpm×5 min to give an oil-in-water emulsion composition. The emulsion particle size of the oil component (a) containing reduced coenzyme Q10 in the oil-in-water emulsion composition was about 0.5 μm. The oil-in-water emulsion composition (75 g) m obtained here was added to oil component (B) heated to 90° C. in advance, which comprised MCT (100 g, Actor manufactured by Riken Vitamin Co., Ltd.), and tetraglycerol pentaoleate (50 g, manufactured by Sakamoto Yakuhin Kogyo Co., Ltd.; SY Glyster PO-3S, HLB 3.0), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The sphericity of the obtained particulate composition was 0.96, volume average particle size was about 200 μm, coenzyme Q content of the particulate composition was 11.9 wt %, and the weight ratio of reduced coenzyme Q10/oxidized coenzyme Q10 was 99.6/0.4. In addition, the average particle size of the domain of oil component (A) in the particulate composition was about 0.5 μm, and it was found that the emulsion particle size of the oil component (a) containing reduced coenzyme Q10 in the oil-in-water emulsion composition in the production step was maintained even after preparation of the particles. In addition, the residual ratio of reduced coenzyme Q10 after preservation of the particulate composition sealed in a glass bottle for 90 days at 40° C. under shading conditions was 98.2%. In addition, the residual ratio of reduced coenzyme Q10 after preservation for 30 days in an environment at 40° C., relative humidity 80% in the air (open) under shading conditions was 99.0%. Moreover, the crystallinity of reduced coenzyme Q10 in the particulate composition as measured by DSC was 0%, and 100 wt % of amorphous or molten reduced coenzyme Q10 was contained.

### Comparative Example 2

Gum arabic (60 g, gum arabic A manufactured by Ina Food Industry Co., Ltd.) was dissolved in distilled water (140 g) at 30° C. to give an aqueous solution of water-soluble excipient. Separately, the reduced coenzyme Q10 powder (9.2 g) obtained in the above-mentioned Production Example 1 and diglycerol monooleate (4.6 g, poem DO-100V manufactured by Riken Vitamin Co., Ltd.) were uniformly mixed at 60° C. to give oil component (a), which was added to an aqueous solution of water-soluble excipient at 60° C., and the mixture was emulsified by TK homomixer Mark II (manufactured by PRIMIX Corporation) at 10000 rpm×5 min to give an oil-in-water emulsion composition. The emulsion particle size of the oil component (a) containing reduced coenzyme Q10 in the oil-in-water emulsion composition was about 0.5 μm. The oil-in-water emulsion composition (75 g) obtained here was added to oil component (B) heated to 90° C. in advance, which comprised MCT (100 g, Actor M-2 manufactured by Riken Vitamin Co., Ltd.), and tetraglycerol pentaoleate (50 g, manufactured by Sakamoto Yakuhin Kogyo Co., Ltd.; SY Glyster PO-3S, HLB 3.0), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The volume average particle size of the obtained particulate composition was about 200 μm, coenzyme Q content of the particulate composition was 12.2 wt %, and the weight ratio of reduced coenzyme Q10/oxidized coenzyme Q10 was 99.1/0.9. In addition, the residual ratio of reduced coenzyme Q10 after preservation of the particulate composition sealed in a glass bottle for 90 days at 40° C. under shading conditions was 97.1%.

### Example 3

Gum arabic (60 g, gum arabic A manufactured by Ina Food Industry Co., Ltd.) and L(+)-ascorbic acid (6.5 g, manufactured by Wako Pure Chemical Industries, Ltd.) were dissolved in distilled water (140 g) at 60° C. to give an aqueous solution of water-soluble excipient. The aqueous solution was maintained at 60° C., enzyme decomposition lecithin (8.0 g, Emultop HL50IP manufactured by Degussa Texturant Systems Japan K.K.) and the reduced coenzyme Q10 powder (31.9 g) obtained in the above-mentioned Production Example 1 were added and dispersed therein, and the dispersion was emulsified by TK homomixer Mark II (manufactured by PRIMIX Corporation) at 10000 rpm×5 min to give an oil-in-water emulsion composition. The emulsion particle size of the oil component (a) containing reduced coenzyme Q10 in the oil-in-water emulsion composition was about 0.5 μm. The oil-in-water emulsion composition (75 g) obtained here was added to oil component (B) heated to 90° C. in advance, which comprised MCT (125 g, Actor M-2 manufactured by Riken Vitamin Co., Ltd.), and tetraglycerol pentaoleate (25 g, manufactured by Sakamoto Yakuhin Kogyo Co., Ltd.; SY Glyster PO-3S, HLB 3.0), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The sphericity of the obtained particulate composition was 0.96, volume average particle size was about 200 μm, coenzyme Q content of the particulate composition was 29.6 wt %, and the weight ratio of reduced coenzyme Q10/oxidized coenzyme Q10 was 99.6/0.4. In addition, the residual ratio of reduced coenzyme Q10 after preservation of the particulate composition sealed in a glass bottle for 90 days at 40° C. under shading conditions was 99.9%. In addition, the residual ratio of reduced coenzyme Q10 after preservation for 30 days in an environment at 40° C., relative humidity 80% in the air (open) under shading conditions was 99.0%. Moreover, the crystallinity of reduced coenzyme Q10 in the particulate composition as measured by DSC was 0%, and 100 wt % of amorphous or molten reduced coenzyme Q10 was contained.

### Comparative Example 3

Gum arabic (60 g, gum arabic A manufactured by Ina Food Industry Co., Ltd.) was dissolved in distilled water (140 g) at 30° C. to give an aqueous solution of water-soluble excipient. The aqueous solution was heated to 60° C., enzyme decomposition lecithin (5.3 g, Emultop HL50IP manufactured by Degussa Texturant Systems Japan K.K.) and the reduced coenzyme Q10 powder (10.6 g) obtained in the above-mentioned Production Example 1 were added and dispersed therein, and the dispersion was emulsified by TK homomixer Mark II (manufactured by PRIMIX Corporation) at 10000 rpm×5 min to give an oil-in-water emulsion composition. The emulsion particle size of the oil component containing reduced coenzyme Q10 in the oil-in-water emulsion composition was about 0.5 μm. The oil-in-water emulsion composition (75 g) obtained here was added to oil component (B) heated to 90° C. in advance, which comprised MCT (125 g, Actor M-2 manufactured by Riken Vitamin Co., Ltd.), and tetraglycerol pentaoleate (25 g, manufactured by Sakamoto Yakuhin Kogyo Co., Ltd.; SY Glyster PO-3S, HLB 3.0), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The volume average particle size of the obtained particulate composition was about 200 μm, coenzyme Q content of the particulate composition was 13.7 wt %, and the weight ratio of reduced coenzyme Q10/oxidized coenzyme Q10 was 99.0/1.0. In addition, the residual ratio of reduced coenzyme Q10 after preservation of the particulate composition sealed in a glass bottle for 90 day at 40° C. under shading conditions was 95.2%.

### Example 4

Gum arabic (60 g, gum arabic A manufactured by Ina Food Industry Co., Ltd.) and L(+)-ascorbic acid (6.5 g, manufactured by Wako Pure Chemical Industries, Ltd.) were dissolved in distilled water (140 g) at 60° C. to give an aqueous solution of water-soluble excipient. The aqueous solution was maintained at 60° C., lecithin (8.0 g, Emulpur IP manufactured by Degussa Texturant Systems Japan K.K.) and the reduced coenzyme Q10 powder (31.9 g) obtained in the above-mentioned Production Example 1 were added and dispersed therein, and the dispersion was emulsified by TK homomixer Mark II (manufactured by PRIMIX Corporation) at 10000 rpm×5 min to give an oil-in-water emulsion composition. The emulsion particle size of the oil component (a) containing reduced coenzyme Q10 in the oil-in-water emulsion composition was about 0.5 μm. The oil-in-water emulsion composition (75 g) obtained here was added to oil component (B) heated to 90° C. in advance, which comprised MCT (125 g, Actor M-2 manufactured by Riken Vitamin Co., Ltd.), and tetraglycerol pentaoleate (25 g, manufactured by Sakamoto Yakuhin Kogyo Co., Ltd.; SY Glyster PO-3S, HLB 3.0), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The sphericity of the obtained particulate composition was 0.96, volume average particle size was about 200 μm, coenzyme Q content of the particulate composition was 29.6 wt %, and the weight ratio of reduced coenzyme Q10/oxidized coenzyme Q10 was 99.5/0.5. In addition, the residual ratio of reduced coenzyme Q10 after preservation of the particulate composition sealed in a glass bottle for 90 days at 40° C. under shading conditions was 98.3%. In addition, the residual ratio of reduced coenzyme Q10 after preservation for 30 days in an environment at 40° C., relative humidity 80% in the air (open) under shading conditions was 99.6%. Moreover, the crystallinity of reduced coenzyme Q10 in the particulate composition as measured by DSC was 0%, and 100 wt % of amorphous or molten reduced coenzyme Q10 was contained.

### Comparative Example 4

Gum arabic (60 g, gum arabic A manufactured by Ina Food Industry Co., Ltd.) was dissolved in distilled water (140 g) at 30° C. to give an aqueous solution of water-soluble excipient. The aqueous solution was heated to 60° C., lecithin (5.3 g, Emulpur IP manufactured by Degussa Texturant Systems Japan K.K.) and the reduced coenzyme Q10 powder (10.6 g) obtained in the above-mentioned Production Example 1 were added and dispersed therein, and the dispersion was emulsified by TK homomixer Mark II (manufactured by PRIMIX Corporation) at 10000 rpm×5 min to give an oil-in-water emulsion composition. The emulsion particle size of the oil component (a) containing reduced coenzyme Q10 in the oil-in-water emulsion composition was about 0.5 μm. The oil-in-water emulsion composition (75 g) obtained here was added to oil component (B) heated to 90° C. in advance, which comprised MCT (125 g, Actor M-2 manufactured by Riken Vitamin Co., Ltd.), and tetraglycerol pentaoleate (25 g, manufactured by Sakamoto Yakuhin Kogyo Co., Ltd.; SY Glyster PO-3S, HLB 3.0), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The volume average particle size of the obtained particulate composition was about 200 μm, coenzyme Q content of the particulate composition was 13.7 wt %, and the weight ratio of reduced coenzyme Q10/oxidized coenzyme Q10 was 99.1/0.9. In addition, the residual ratio of reduced coenzyme Q10 after preservation of the particulate composition sealed in a glass bottle for 90 days at 40° C. under shading conditions was 94.6%.

### Example 5

Gum arabic (60 g, gum arabic A manufactured by Ina Food Industry Co., Ltd.) and L(+)-ascorbic acid (6.0 g, manufactured by Wako Pure Chemical Industries, Ltd.) were dissolved in distilled water (140 g) at 60° C. to give an aqueous solution of water-soluble excipient. The aqueous solution was maintained at 60° C., diglycerol monooleate (4.0 g, poem DO-100V manufactured by Riken Vitamin Co., Ltd.) and the reduced coenzyme Q10 powder (30.0 g) obtained in the above-mentioned Production Example 1 were added and dispersed therein, and the dispersion was emulsified by TK homomixer Mark II (manufactured by PRIMIX Corporation) at 10000 rpm×5 min to give an oil-in-water emulsion composition. The emulsion particle size of the oil component (a) containing reduced coenzyme Q10 in the oil-in-water emulsion composition was about 0.5 μm. The oil-in-water emulsion composition (75 g) obtained here was added to oil component (B) heated to 90° C. in advance, which comprised MCT (148 g, Actor M-2 manufactured by Riken Vitamin Co., Ltd.), and enzyme decomposition lecithin (2 g, Emultop HL50IP manufactured by Degussa Texturant Systems Japan K.K.), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The sphericity of the obtained particulate composition was 0.96, volume average particle size was about 200 μm, coenzyme Q content of the particulate composition was 29.6 wt %, and the weight ratio of reduced coenzyme Q10/oxidized coenzyme Q10 was 99.6/0.4. In addition, the residual ratio of reduced coenzyme Q10 after preservation of the particulate composition for 30 days in an environment at 40° C., relative humidity 80% in the air (open) under shading conditions was 99.2%. Moreover, the crystallinity of reduced coenzyme Q10 in the particulate to composition as measured by DSC was 0%, and 100 wt % of amorphous or molten reduced coenzyme Q10 was contained.

### Comparative Example 5

Gum arabic (60 g, gum arabic A manufactured by Ina Food Industry Co., Ltd.) was dissolved in distilled water (140 g) at 60° C. to give an aqueous solution of water-soluble excipient. The aqueous solution was maintained at 60° C., L(+)-Ascorbyl palmitate (6.0 g, manufactured by Wako Pure Chemical Industries, Ltd.), diglycerol monooleate (4.0 g, poem DO-100V manufactured by Riken Vitamin Co., Ltd.) and the reduced coenzyme Q10 powder (30.0 g) obtained in the above-mentioned Production Example 1 were added and dispersed therein, and the dispersion was emulsified by TK homomixer Mark II (manufactured by PRIMIX Corporation) at 10000 rpm×5 min to give an oil-in-water emulsion composition. The emulsion particle size of the oil component (a) containing reduced coenzyme Q10 in the oil-in-water emulsion composition was about 0.5 μm. The oil-in-water emulsion composition (75 g) obtained here was added to oil component (B) heated to 90° C. in advance, which comprised MCT (148 g, Actor M-2 manufactured by Riken Vitamin Co., Ltd.), and enzyme decomposition lecithin (2 g, Emultop HL50IP manufactured by Degussa Texturant Systems Japan K.K.), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The volume average particle size of the obtained particulate composition was about 200 μm, coenzyme Q content of the particulate composition was 29.6 wt %, and the weight ratio of reduced coenzyme Q10/oxidized coenzyme Q10 was 99.5/0.5. In addition, the residual ratio of reduced coenzyme Q10 after preservation for 30 days in an environment at 40° C., relative humidity 80% in the air (open) under shading conditions was 95.7%.

### Comparative Example 6

Gum arabic (60 g, gum arabic A manufactured by Ina Food Industry Co., Ltd.) was dissolved in distilled water (140 g) at 60° C. to give an aqueous solution of water-soluble excipient. The aqueous solution was maintained at 60° C., diglycerol monooleate (4.0 g, poem DO-100V manufactured by Riken Vitamin Co., Ltd.) and the reduced coenzyme Q10 powder (30.0 g) obtained in the above-mentioned Production Example 1 were added and dispersed therein, and the dispersion was emulsified by TK homomixer Mark II (manufactured by PRIMIX Corporation) at 10000 rpm×5 min to give an oil-in-water emulsion composition. The emulsion particle size of the oil component (a) containing reduced coenzyme Q10 in the oil-in-water emulsion composition was about 0.5 μm. The oil-in-water emulsion composition (75 g) obtained here was added to oil component (B) heated to 90° C. in advance, which comprised MCT (148 g, Actor M-2 manufactured by Riken Vitamin Co., Ltd.), and enzyme decomposition lecithin (2 g, Emultop HL50IP manufactured by Degussa Texturant Systems Japan K.K.), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The volume average particle size of the obtained particulate composition was about 200 μm, coenzyme Q content of the particulate composition was 31.4 wt %, and the weight ratio of reduced coenzyme Q10/oxidized coenzyme Q10 was 99.0/1.0. In addition, the residual ratio of reduced coenzyme Q10 after preservation for 30 days in an environment at 40° C., relative humidity 80% in the air (open) under shading conditions was 88.4%.

### Example 6

Gum arabic (60 g, gum arabic A manufactured by Ina Food Industry Co., Ltd.) and L(+)-ascorbic acid (6.0 g, manufactured by Wako Pure Chemical Industries, Ltd.) were dissolved in distilled water (140 g) at 60° C. to give an aqueous solution of water-soluble excipient. The aqueous solution was maintained at 60° C., enzyme decomposition lecithin (2.0 g, Emultop HL50IP manufactured by Degussa Texturant Systems Japan K.K.) and the reduced coenzyme Q10 powder (30.0 g) obtained in the above-mentioned Production Example 1 were added and dispersed therein, and the dispersion was emulsified by TK homomixer Mark II (manufactured by PRIMIX Corporation) at 10000 rpm×5 min to give an oil-in-water emulsion composition. The emulsion particle size of the oil component (a) containing reduced coenzyme Q10 in the oil-in-water emulsion composition was about 0.5 μm. The oil-in-water emulsion composition (75 g) obtained here was added to oil component (B) heated to 90° C. in advance, which comprised MCT (148 g, Actor M-2 manufactured by Riken Vitamin Co., Ltd.), and enzyme decomposition lecithin (2 g, Emultop HL50IP manufactured by Degussa Texturant Systems Japan K.K.), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The sphericity of the obtained particulate composition was 0.96, and volume average particle size was about 200 μm. In addition, the average particle size of the domain of oil component (A) in the particulate composition was about 0.5 μm, and it was found that the emulsion particle size of the oil component (a) containing reduced coenzyme Q10 in the oil-in-water emulsion composition in the production step was maintained even after preparation of the particles. The content of coenzyme Q in the obtained particulate composition was 29.5 wt %, and the weight ratio of reduced coenzyme Q10/oxidized coenzyme Q10 was 99.7/0.3. In addition, the residual ratio of reduced coenzyme Q10 after preservation of the particulate composition for 30 days in an environment at 40° C., relative humidity 80% in the air (open) under shading conditions was 99.0%. Moreover, the crystallinity of reduced coenzyme Q10 in the particulate composition as measured by DSC was 0%, and 100 wt % of amorphous or molten reduced coenzyme Q10 was contained.

### Comparative Example 7

Gum arabic (60 g, gum arabic A manufactured by Ina Food Industry Co., Ltd.) was dissolved in distilled water (140 g) at 60° C. to give an aqueous solution of water-soluble excipient. The aqueous solution was maintained at 60° C., L(+)-Ascorbyl palmitate (6.0 g, manufactured by Wako Pure Chemical Industries, Ltd.), enzyme decomposition lecithin (2.0 g, Emultop HL50IP manufactured by Degussa Texturant Systems Japan K.K.) and the reduced coenzyme Q10 powder (30.0 g) obtained in the above-mentioned Production Example 1 were added and dispersed therein, and the dispersion was emulsified by TK homomixer Mark II (manufactured by PRIMIX Corporation) at 10000 rpm×5 min to give an oil-in-water emulsion composition. The emulsion particle size of the oil component (a) containing reduced coenzyme Q10 in the oil-in-water emulsion composition was about 0.5 μm. The oil-in-water emulsion composition (75 g) obtained here was added to oil component (B) heated to 90° C. in advance, which comprised MCT (148 g, Actor M-2 manufactured by Riken Vitamin Co., Ltd.), and enzyme decomposition lecithin (2 g, Emultop HL50IP manufactured by Degussa Texturant Systems Japan K.K.), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The volume average particle size of the obtained particulate composition was about 200 μm, coenzyme Q content of the particulate composition was 29.5 wt %, and the weight ratio of reduced coenzyme Q10/oxidized coenzyme Q10 was 99.4/0.6. In addition, the residual ratio of reduced coenzyme Q10 after preservation for 30 days in an environment at 40° C., relative humidity 80% in the air (open) under shading conditions was 97.5%.

### Comparative Example 8

Gum arabic (60 g, gum arabic A manufactured by Ina Food Industry Co., Ltd.) was dissolved in distilled water (140 g) at 60° C. to give an aqueous solution of water-soluble excipient. The aqueous solution was maintained at 60° C., enzyme decomposition lecithin (2.0 g, Emultop HL50IP manufactured by Degussa Texturant Systems Japan K.K.) and the reduced coenzyme Q10 powder (30.0 g) obtained in the above-mentioned Production Example 1 were added and dispersed therein, and the dispersion was emulsified by TK homomixer Mark II (manufactured by PRIMIX Corporation) at 10000 rpm×5 min to give an oil-in-water emulsion composition. The emulsion particle size of the oil component (a) containing reduced coenzyme Q10 in the oil-in-water emulsion composition was about 0.5 p.m. The oil-in-water emulsion composition (75 g) obtained here was added to oil component (B) heated to 90° C. in advance, which comprised MCT (148 g, Actor M-2 manufactured by Riken Vitamin Co., Ltd.), and enzyme decomposition lecithin (2 g, Emultop HL50IP manufactured by Degussa Texturant Systems Japan K.K.), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The volume average particle size of the obtained particulate composition was about 200 μm, coenzyme Q content of the particulate composition was 31.9 wt %, and the weight ratio of reduced coenzyme Q10/oxidized coenzyme Q10 was 99.1/0.9. In addition, the residual ratio of reduced coenzyme Q10 after preservation for 30 days in an environment at 40° C., relative humidity 80% in the air (open) under shading conditions was 88.2%.

### Example 7

Gum arabic (53 g, gum arabic A manufactured by Ina Food Industry Co., Ltd.) and L(+)-ascorbic acid (6.0 g, manufactured by Wako Pure Chemical Industries, Ltd.) and dextrin (11 g, Pinedex #100 manufactured by Matsutani Chemical Industry Co., Ltd.) were dissolved in distilled water (140 g) at 60° C. to give an aqueous solution of water-soluble excipient. The aqueous solution was maintained at 60° C., the reduced coenzyme Q10 powder (28 g) obtained in the above-mentioned Production Example 1 and oxidized coenzyme Q10 (2.0 g, manufactured by Kaneka Corporation) were added and dispersed therein (weight ratio of reduced coenzyme Q10 and oxidized coenzyme Q10: 92.4/7.6), and the dispersion was emulsified by TK homomixer Mark II (manufactured by PRIMIX Corporation) at 10000 rpm×5 min to give an oil-in-water emulsion composition. The emulsion particle size of the oil component (a) containing coenzyme Q10 in the oil-in-water emulsion composition was about 0.5 μm. The oil-in-water emulsion composition (75 g) obtained here was added to oil component (B) heated to 90° C. in advance, which comprised MCT (148 g, Actor M-2 manufactured by Riken Vitamin Co., Ltd.) and enzyme decomposition lecithin (2 g, Emultop HL50IP manufactured by Degussa Texturant Systems Japan K.K.), and the number of stirring rotation was adjusted so that the oil-in-water emulsion composition would be suspended in the oily substance (B). The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The sphericity of the obtained particulate composition was 0.96, volume average particle size was about 200 μm, absolute specific gravity of whole particulate composition was 1.30, and absolute specific gravity value of the component water-soluble excipient to be calculated was 1.46. In addition, the average particle size of the domain of oil component (A) in the particulate composition was about 0.5 μm, and it was found that the emulsion particle size of the oil component (a) containing reduced coenzyme Q10 in the oil-in-water emulsion composition in the production step was maintained even after preparation of the particles. Furthermore, the coenzyme Q content of the particulate composition was 29.8 wt %, and the weight ratio of reduced coenzyme Q10/oxidized coenzyme Q10 was 99.3/0.7, thus showing an increased ratio of reduced coenzyme Q10 as compared to the weight ratio used during production.

In addition, the residual ratio of reduced coenzyme Q10 after preservation of the particulate composition sealed in a glass bottle for 90 days at 40° C. under shading conditions was 98.3%. In addition, the residual ratio of reduced coenzyme Q10 after preservation for 30 days in an environment at 40° C., relative humidity 80% in the air (open) under shading conditions was 98.5%. Moreover, the crystallinity of reduced coenzyme Q10 in the particulate composition as measured by DSC was 0%, and 100 wt % of amorphous or molten reduced coenzyme Q10 was contained.

### Comparative Example 9

Gum arabic (53 g, gum arabic A manufactured by Ina Food Industry Co., Ltd.) and dextrin (11 g, Pinedex #100 manufactured by Matsutani Chemical Industry Co., Ltd.) were dissolved in distilled water (140 g) at 60° C. to give an aqueous solution of water-soluble excipient. The aqueous solution was maintained at 60° C., the reduced coenzyme Q10 powder (28 g) obtained in the above-mentioned Production Example 1 and oxidized coenzyme Q10 (2.0 g, manufactured by Kaneka to Corporation) (weight ratio of reduced coenzyme Q10 and oxidized coenzyme Q10: 92.4/7.6) were added and dispersed therein, and the dispersion was emulsified by TK homomixer Mark II (manufactured by PRIMIX Corporation) at 10000 rpm×5 min to give an oil-in-water emulsion composition. The emulsion particle size of the oil component (a) containing coenzyme Q10 in the oil-in-water emulsion composition was about 0.5 μm. The oil-in-water emulsion composition (75 g) obtained here was added to oil component (B) heated to 90° C. in advance, which comprised MCT (148 g, Actor M-2 manufactured by Riken Vitamin Co., Ltd.) and enzyme decomposition lecithin (2 g, Emultop HL50IP manufactured by Degussa Texturant Systems Japan K.K.), and the number of stirring rotation was adjusted to the same conditions as in Example 7. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The sphericity of the obtained particulate composition was 0.96, volume average particle size was about 200 μm, coenzyme Q content of the particulate composition was 31.2 wt %, and the weight ratio of reduced coenzyme Q10/oxidized coenzyme Q10 was 91.7/8.3. In addition, the residual ratio of reduced coenzyme Q10 after preservation of the particulate composition sealed in a glass bottle for 90 days at 40° C. under shading conditions was 97.9%. In addition, the residual ratio of reduced coenzyme Q10 after preservation for 30 days in an environment at 40° C., relative humidity 80% in the air (open) under shading conditions was 71.9%.

### Example 8

Gum arabic (70 g, gum arabic A manufactured by Ina Food Industry Co., Ltd.), L(+)-ascorbic acid (10 g, manufactured by Wako Pure Chemical Industries, Ltd.) and sucrose (10 g, manufactured by Wako Pure Chemical Industries, Ltd.) were dissolved in distilled water (140 g) at 60° C. to give an aqueous solution of water-soluble excipient. The aqueous solution was maintained at 60° C., the reduced coenzyme Q10 powder (8.0 g) obtained in the above-mentioned Production Example 1 and oxidized coenzyme Q10 (2.0 g, manufactured by Kaneka Corporation) (weight ratio of reduced coenzyme Q10 and oxidized coenzyme Q10: 92.7/7.3) were added and dispersed therein, and the dispersion was emulsified by TK homomixer Mark II (manufactured by PRIMIX Corporation) at 10000 rpm×5 min to give an oil-in-water emulsion composition. The emulsion particle size of the oil component (a) containing coenzyme Q10 in the oil-in-water emulsion composition was about 0.5 μm. The oil-in-water emulsion composition (75 g) obtained here was added to oil component (B) heated to 90° C. in advance, which comprised MCT (148 g, Actor M-2 manufactured by Riken Vitamin Co., Ltd.), and enzyme decomposition lecithin (2 g, Emultop HL50IP manufactured by Degussa Texturant Systems Japan K.K.), and the mixture was stirred. The temperature of the suspension was adjusted to 105° C. with continuous stirring, whereby water was removed from oil-in-water emulsion composition suspension droplets. As a result, water was mostly evaporated in about 30 min. Thereafter, oil component (B) was collected by filtration by solid-liquid separation according to a conventional method. The oil component (B) attached to the particles was washed with about 500 g of ethanol and dried at 50° C. to give a particulate composition containing reduced coenzyme Q10.

The sphericity of the obtained particulate composition was 0.96, the volume average particle size was about 200 μm, the coenzyme Q content of the particulate composition was 9.9% wt %, and the weight ratio of reduced coenzyme Q10/oxidized coenzyme Q10 was 99.3/0.7, thus showing an increased ratio of reduced coenzyme Q10 as compared to the weight ratio used during production.

### Comparative Example 9

The white dry crystals of reduced coenzyme Q10 obtained in Production Example 1 was pulverized in a mortar to give a reduced coenzyme Q10 powder. The residual ratio of reduced coenzyme Q10 was 28% after the obtained reduced coenzyme Q10 powder was preserved for 30 days at 40° C. under shading conditions.

### Example 9

Gum arabic (80 g, gum arabic A manufactured by Ina Food Industry Co., Ltd.) and L-ascorbic acid (10 g) were dissolved in distilled water (300 g) at 30° C. to give an aqueous solution of water-soluble excipient. The aqueous solution was heated to 60° C., the reduced coenzyme Q10 powder (10 g) obtained in the above-mentioned Production Example 1 was added and melted, and the mixture was emulsified by TK homomixer Mark II (manufactured by PRIMIX Corporation) at 10000 rpm×5 min to give an oil-in-water emulsion composition. The emulsified particle size (average particle size of domain) of the reduced coenzyme Q10 in the oil-in-water emulsion composition was about 1 μm. The oil-in-water emulsion composition was spray dried under the conditions of hot air temperature at 200° C. with a spray dryer (B-290 manufactured by Nihon BUCHI K.K.) to give a particulate composition containing reduced coenzyme Q10.

The obtained particulate composition was sphericity; 0.85, and volume average particle size; 7.1 μm. The coenzyme Q content of the particulate composition was 10.0 wt %, and the weight ratio of reduced coenzyme Q10/oxidized coenzyme Q10 was 99.6/0.4.

### Example 10

### Oral Absorbability Test

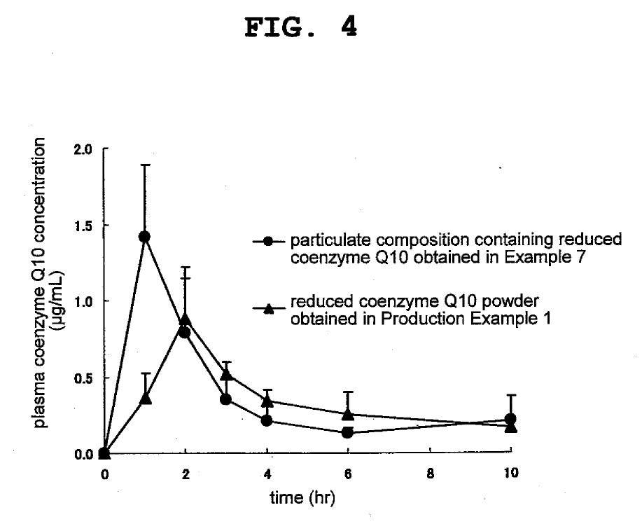

The particulate composition containing reduced coenzyme Q10 obtained in Example 7 and the reduced coenzyme Q10 powder obtained in Production Example 1 were each filled in a gelatin hard capsule, and orally administered to 14-week-old male Sprague-Dawley rats (purchased from: Japan SLC, Inc) at a dose of 10 mg/kg based on reduced coenzyme Q10. Blood was taken from each rat after 1, 2, 3, 4, 6 and 10 hours from the administration of the test substance. The collected blood was centrifuged to give plasma. Then, reduced coenzyme Q10 in the plasma was oxidized, oxidized coenzyme Q10 therein was extracted, and the coenzyme Q10 concentration of the plasma was measured as oxidized coenzyme Q10 by HPLC. The results are shown in FIG. 4.

As shown in FIG. 4, when a particulate composition containing reduced coenzyme Q10 of the present invention was ingested, the coenzyme Q10 concentration in the plasma was found to increase more rapidly and more greatly after ingestion as compared to simple ingestion of reduced coenzyme

Q10 powder. In other words, from the above-mentioned results, it was found that a particulate composition containing reduced coenzyme Q10 of the present invention simultaneously shows high stability, high oral absorbability and rapid absorbability.

### Example 11

### Soft Capsule

The particulate composition containing reduced coenzyme Q10 obtained in the above-mentioned Example 3 was added to a mixture of safflower oil and beeswax to give a slurry, which was filled in a capsule according to a conventional method to give a gelatin soft capsule preparation containing the following components.

| | |
|---|---|
| particulate composition containing | 30 parts by weight |
| reduced coenzyme Q10 | |
| safflower oil | 65 parts by weight |
| beeswax | 5 parts by weight. |

### Example 12

### Hard Capsules

A particulate composition containing reduced coenzyme Q10 obtained in the above-mentioned Example 3 was singly filled in a hard capsule according to a conventional method to give a hard capsule preparation. The hard capsule was preserved for 90 days in an environment at 40° C. in the air (open) under shading conditions. The residual ratio of reduced coenzyme Q10 in the preparation in the capsule after preservation was 99.9%.

### Comparative Example 10

The reduced coenzyme Q10 (30 parts by weight) obtained in the above-mentioned Production Example 1 and gum arabic (60 parts by weight, gum arabic A manufactured by Ina Food Industry Co., Ltd.) were mixed and filled in a hard capsule according to a conventional method to give a hard capsule preparation. The hard capsule was preserved for 90 days in an environment at 40° C. in the air (open) under shading conditions. The residual ratio of reduced coenzyme Q10 in the preparation in the capsule after preservation was 31.3%.

### Example 13

### Tablet

A particulate composition containing reduced coenzyme Q10 obtained in the above-mentioned Example 3 was mixed with crystalline cellulose (Avicel), and further with magnesium stearate and a tablet containing the following components was obtained according to a conventional method. The appearance of the obtained tablet was good with no tableting trouble, and no trouble occurred during production (attachment of drug to surface of die or punch) and the like.

| | |
|---|---|
| particulate composition containing | 50 parts by weight |
| reduced coenzyme Q10 | |
| crystalline cellulose | 50 parts by weight |
| magnesium stearate | 1 part by weight. |

### Example 14

### Chewable Tablet

The particulate composition containing reduced coenzyme Q10 obtained in the above-mentioned Example 7 was mixed with reduction malt sugar, crystalline cellulose (Avicel), glucose, lactose and magnesium stearate according to a conventional method to give a tablet (chewable tablet) containing the following components. The appearance of the obtained tablet was good with no tableting trouble, and no trouble occurred during production (attachment of drug to surface of die or punch) and the like.

| | |
|---|---|
| particulate composition containing | 10 parts by weight |
| reduced coenzyme Q10 | |
| reduction malt sugar | 40 parts by weight |
| crystalline cellulose | 4 parts by weight |
| glucose | 40 parts by weight |
| lactose | 4 parts by weight |
| magnesium stearate | 1 part by weight. |

The chewable tablet was preserved for 30 days in an environment at 25° C., relative humidity 60% in the air (open) under shading conditions. The residual ratio of reduced coenzyme Q10 in the preparation after preservation was 99.0%.

### Comparative Example 11

The reduced coenzyme Q10 obtained in the above-mentioned Production Example 1 was mixed with reduction malt sugar, crystalline cellulose (Avicel), glucose, lactose and magnesium stearate according to a conventional method to give a tablet (chewable tablet) containing the following components.

| | |
|---|---|
| reduced coenzyme Q10 | 10 parts by weight |
| reduction malt sugar | 40 parts by weight |
| crystalline cellulose | 4 parts by weight |
| glucose | 40 parts by weight |
| lactose | 4 parts by weight |
| magnesium stearate | 1 part by weight. |

The chewable tablet was preserved for 30 days in an environment at 25° C., relative humidity 60% in the air (open) under shading conditions. The residual ratio of reduced coenzyme Q10 in the preparation after preservation was 70.3%.

### Example 15

### Drinks

To a commercially available oolong tea (500 mL) was added and mixed a particulate composition containing reduced coenzyme Q10 obtained in Example 7 (100 mg) to disperse the particulate composition. As a result, the matrix component in the particulate composition was dissolved, and the oolong tea containing reduced coenzyme Q10 was obtained, wherein the oil component containing reduced coenzyme Q10 was emulsified. The emulsion particle size of the oil component containing reduced coenzyme Q10 was about 0.5 μm.

### Example 16

Gum arabic (57 g, gum arabic A manufactured by Ina Food Industry Co., Ltd.) and L(+)-ascorbic acid (8.0 g, manufactured by Wako Pure Chemical Industries, Ltd.) were dissolved in distilled water (140 g) at 60° C. to give an aqueous solution of water-soluble excipient. The aqueous solution was maintained at 60° C., oxidized coenzyme Q10 (35 g, manufactured by Kaneka Corporation) was added and melted therein. The mixture was stirred to give an oil-in-water emulsion composition, and further stirred at 80° C. for 10 hr to perform reduction reaction while maintaining the emulsion state. The weight ratio of reduced coenzyme Q10/oxidized coenzyme Q10 after the reaction was 99.4/0.6.

### Example 17

Gum arabic (60 g, gum arabic A manufactured by Ina Food Industry Co., Ltd.) and L(+)-ascorbic acid (5.0 g, manufactured by Wako Pure Chemical Industries, Ltd.) were dissolved in distilled water (140 g) at 60° C. to give an aqueous solution of water-soluble excipient. The aqueous solution was maintained at 60° C., oxidized coenzyme Q10 (5 g, manufactured by Kaneka Corporation) and reduced coenzyme Q10 (30 g) prepared in Production Example 1 were added and melted therein. The weight ratio of reduced coenzyme Q10/oxidized coenzyme Q10 was 85.3/14.7 then. The mixture was stirred to give an oil-in-water emulsion composition, which was stirred at 80° C. for 5 hr to perform reduction reaction while maintaining the emulsion state. The weight ratio of reduced coenzyme Q10/oxidized coenzyme Q10 after the reaction was 99.5/0.5.

From the above-mentioned Examples and Comparative Examples, it is clear that the particulate composition of the present invention containing water-soluble ascorbic acid in a matrix containing a water-soluble excipient as a main component shows improved oxidative stability of reduced coenzyme Q10 contained in the particulate composition, even when a solvent component such as fats and oils, emulsifier, ethanol and the like is not at all present or completely insufficient to achieve compatibility of reduced coenzyme Q10 and ascorbic acids at a molecule level. Furthermore, it is clear that the composition maintains extremely high oxidative stability even under high humidity condition with relative humidity of 80%.

1. A particulate composition comprising an oil component (A) comprising reduced coenzyme Q10, and a matrix comprising a water-soluble excipient and a water-soluble ascorbic acid, wherein the oil component (A) is polydispersed forming a domain in the matrix.

2. The particulate composition of claim 1, wherein the water-soluble ascorbic acid is at least one kind selected from the group consisting of ascorbic acid, rhamno-ascorbic acid, arabo-ascorbic acid, gluco-ascorbic acid, fuco-ascorbic acid, glucohepto-ascorbic acid, xylo-ascorbic acid, galacto-ascorbic acid, gulo-ascorbic acid, allo-ascorbic acid, erythro-ascorbic acid, 6-desoxyascorbic acid and a salt thereof.

3. The particulate composition of claim 1, which has a sphericity of not less than 0.8.

4. The particulate composition of claim 1, wherein not less than 10 wt % of the reduced coenzyme Q10 in the particulate composition is non-crystalline.

5. The particulate composition of claim 1, wherein the reduced coenzyme Q10 and the water-soluble ascorbic acid are contained in the particulate composition at a weight ratio of 100:1-1:5.

6. The particulate composition of claim 1, wherein the water-soluble excipient is at least one kind selected from the group consisting of a water-soluble polymer, surfactant (C), sugar and a yeast cell wall.

7. The particulate composition of claim 6, wherein the water-soluble polymer is at least one kind selected from the group consisting of gum arabic, gelatin, agar, starch, pectin, carageenan, casein, dried albumen, curdlan, alginic acids, soybean polysaccharide, pullulan, celluloses, xanthan gum, carmellose salt and polyvinylpyrrolidone.

8. The particulate composition of claim 6, wherein the surfactant (C) is at least one kind selected from the group consisting of glycerol fatty acid ester, sucrose fatty acid ester, sorbitan fatty acid ester, polyoxyethylenesorbitan fatty acid ester, lecithins and saponins.

9. The particulate composition of claim 6, wherein the sugar is at least one kind selected from the group consisting of monosaccharide, disaccharide, oligosaccharide, sugar alcohol and polysaccharide.

10. The particulate composition of claim 1, wherein the oil component (A) comprises 5-100 wt % of coenzyme Q10, 0-95 wt % of fat and oil, and 0-95 wt % of surfactant (D).

11. The particulate composition of claim 10, wherein the surfactant (D) is at least one kind selected from the group consisting of glycerol fatty acid ester, polyglycerol ester, sucrose fatty acid ester, sorbitan fatty acid ester, polyoxyethylenesorbitan fatty acid ester and propylene glycol fatty acid ester, each having an HLB of not more than 10, and lecithins.

12. The particulate composition of claim 1, wherein the content of the reduced coenzyme Q10 in the particulate composition is 1-70 wt %.

13. The particulate composition of claim 1, wherein the domain formed by the oil component (A) has an average particle size of 0.01-50 μm.

14. A method of producing a particulate composition comprising reduced coenzyme Q10, which comprises preparing an oil-in-water emulsion composition from an oil component (a) containing coenzyme Q10 and an aqueous solution comprising water-soluble ascorbic acid and a water-soluble excipient, and then removing water in the oil-in-water emulsion composition.

15. The method of claim 14, wherein the coenzyme Q10 contained in the oil component (a) is reduced coenzyme Q10.

16. The method of claim 14, wherein oxidized coenzyme Q10 or a mixture of oxidized coenzyme Q10 and reduced coenzyme Q10 is used as the coenzyme Q10 to be contained in the oil component (a), and at least a part of oxidized coenzyme Q10 is reduced into reduced coenzyme Q10, during the production process of the particulate composition.

17. The method of claim 14, wherein the water-soluble ascorbic acid is at least one kind selected from the group consisting of ascorbic acid, rhamno-ascorbic acid, arabo-ascorbic acid, gluco-ascorbic acid, fuco-ascorbic acid, glucohepto-ascorbic acid, xylo-ascorbic acid, galacto-ascorbic acid, gulo-ascorbic acid, allo-ascorbic acid, erythro-ascorbic acid, 6-desoxyascorbic acid and a salt thereof.

18. The production method of claim 14, wherein the oil-in-water emulsified composition is suspended in the oil component (B), and thereafter the water in the emulsified composition is removed in the oil component (B).

19. The production method of claim 18, wherein the oil component (B) comprises 5-99.99 wt % of fat and oil and 0.01-95 wt % of surfactant (E).

20. The production method of claim 19, wherein the surfactant (E) is at least one kind selected from the group consisting of glycerol fatty acid ester, polyglycerol ester, sucrose fatty acid ester, sorbitan fatty acid ester and polyoxyethylenesorbitan fatty acid ester, each having an HLB of not more than 10, and lecithins.

21. The method of claim 14, wherein the water in the oil-in-water emulsion composition is removed by spray drying the oil-in-water emulsion composition in a gaseous phase.

22. The production method of claim 14, wherein the obtained particulate composition has a sphericity of not less than 0.8

23. The production method of claim 14, wherein 1-500 parts by weight of water-soluble ascorbic acid is used relative to 100 parts by weight of coenzyme Q10.

24. A method of producing reduced coenzyme Q10, comprising preparing an oil-in-water emulsion composition from an oil component (a) comprising coenzyme Q10, and an aqueous solution comprising water-soluble ascorbic acid and a water-soluble excipient, and reducing oxidized coenzyme Q10 of coenzyme Q10 in the oil-in-water emulsion composition.

25. The production method of claim 24, wherein the water-soluble excipient is at least one kind selected from the group consisting of gum arabic, gelatin, agar, starch, pectin, carageenan, casein, dried albumen, curdlan, alginic acids, soybean polysaccharides, pullulan, celluloses, xanthan gum, carmellose salt, polyvinylpyrrolidone, sugar and yeast cell wall.

26. A method of improving absorption of coenzyme Q10 in the body, comprising administering the particulate composition of claim 1 to a subject of administration.

27. The method of claim 26, wherein the subject of administration is an athlete or a user of an enteral feeding product.
